(19) United States
(12) Patent Application Publication
(10) Pub. No.: US 2012/0239136 A1
(43) Pub. Date: Sep. 20, 2012

(54) FLEXIBLE INTRALUMINAL STENT

(75) Inventor: Mark Bruzzi, Co. Galway (IE)

(73) Assignee: NATIONAL UNIVERSITY OF IRELAND, GALWAY, Galway (IE)

(21) Appl. No.: 13/319,322

(22) PCT Filed: May 7, 2010
(86) PCT No.: PCT/EP2010/056302

§ 371 Date: May 31, 2012

(30) Foreign Application Priority Data
May 8, 2009 (IE) 2009/0364

(51) Int. Cl. A61F 2/82

(52) U.S. Cl. 623/1.16; 623/1.15

(57) ABSTRACT

A stent comprising: at least one individual closed hoop member formed from a resilient material, the hoop, in a two dimensional configuration, having a diametral dimension that exceeds the diameter of the largest blood vessel into which it will be inserted; and the hoop being resiliency deformable or resiliency deformed so as to occupy a spatial envelope of reduced diameter when inserted in a lumen, wherein when occupying the spatial envelope of reduced diameter, the closed hoop member has at least two substantially diametrically-opposed generally circumferentially extending regions; and at least two regions that extend at least in part generally axially relative to the generally circumferentially extending regions, each of said regions that extend at least in part generally axially being disposed between two of said generally circumferentially extending regions.

### FIELD OF THE INVENTION

This invention is related to the field of medical devices, and in particular to stents. The stents of the invention are adapted to hold the lumen of a blood vessel open, or to hold another medical device in place within the lumen of a blood vessel or chamber. The stent is particularly well adapted for use in peripheral arteries.

### BACKGROUND TO THE INVENTION

Percutaneous angioplasty and stenting is the non-invasive treatment of choice for the occlusion of arteries. Stenting is primarily used to treat occlusion of the coronary artery, with high success rates. These high success rates have led to the implantation of stents into other areas of the body susceptible to occlusion, such as the carotid artery and the superficial femoral artery (SFA).

However, the success rates of coronary stents have not translated for peripheral arteries with lower patency rates depending on the location and type of occlusion. Failure in the stenting of peripheral arteries occurs for many reasons including restenosis (re-occlusion of an artery due to intimal hyperplasia), luminal thrombosis (due to non-laminar haemodynamic blood flow and/or exposure of non-biocompatible material to blood), stent kinking and fracture (for example, due to repetitive loading in vivo), vessel kinking, rupture, failed access, and stent migration.

In order to understand why the success rates differ, it is important to appreciate the movements and forces exerted on these arteries. The coronary artery is located on the surface of the heart. Apart from the systolic and diastolic pressures, the artery only undergoes slight elongation and compression as the heart beats. In comparison, a peripheral artery has a more complex set of movements. For example, the SFA is subjected to external forces such as compression, torsion, and elongation during normal gait. The carotid artery also undergoes similar motion due to movement of the head. It has been proposed that this combination of motion and forces results in higher failure rates in stented peripheral arteries. Such failure may be due to an accelerated healing response induced by blood vessel wall injury, which causes neointimal hyperplasia. Additionally, the size of the lumens may vary significantly and as such, are difficult to stent.

Conventional stent designs consist of a semi-rigid dense metal skeleton. As a result, the stent may restrict the extension, compression, rotation, or bending of the lumen hence increasing the stresses/strains which may result in increased injury. Additionally, relative displacement between the stent and the lumen may cause such injury. This is primarily due to the axially connected cellular structure of peripheral stents currently on the market. Stents that explicitly target the challenging conditions that the SFA demands, include the SUPERA® stent developed by IDev Inc., the Protégé® Everflex device developed by ev3™, the SMART® sent developed by Cordis Corporation, the WALLSTENT® RX Endoprosthesis developed by Boston Scientific Corporation, the GORE VIABHAN® Endoprosthesis developed by W. L. Gore and Associates, and the Life® stent (C. R. Bard, Incorporated). All of these stents vary in their design and claims of flexibility and resistance to fatigue failure. The market has indeed welcomed this, as fatigue fracture has been a great concern for failure in stenting peripheral arteries.

The SUPERA® stent (IDev Inc.) is a self-expanding wire interwoven nitinol (titanium/nickel alloy) tube stent. The Protégé® Everflex device (ev3™) is a self-expanding nitinol stent system, which is cut from a nitinol tube in an open lattice design, and has tantalum radiopaque markers at the proximal and distal ends of the stent. The SMART® stent (Cordis Corporation) is laser cut from a solid nitinol tube into a fine mesh (“Z” configuration) design. The WALLSTENT® RX Endoprosthesis (Boston Scientific Corporation) is a tubular mesh constructed from a biomedical super alloy wire with a tantalum core for visibility under fluoroscopy. The GORE VIABHAN® Endoprosthesis stent (W. L. Gore and Associates) is constructed with an expanded polytetrafluoroethylene (ePTFE) liner attached to an external nitinol stent structure. The Life® stent (C. R. Bard, Incorporated) is a self-expanding helical tubular stent.

However, a number of the above-referenced stent designs include significant axial and torsional stiffness due to the presence of axial structural members. These semi-rigid stent designs in peripheral blood vessels, when subjected to high in vivo deformation, may promote blood vessel injury due to the stent “grating” or scraping against the artery wall resulting in intimal hyperplasia and restenosis.

In addition, high stresses within the stented artery due to such stiff stents are also responsible for stent migration and stent fractures of varying degrees. It has already been documented that the abrupt compliance mismatch that exists at the junction between the stent ends and the host arterial wall disturbs both the vascular haemodynamics and the natural wall stress distribution.

The disclosed present invention, which involves the use of multi-segmented stent technology, has the capability to exceed the performance of the marketed flexible peripheral vascular stents, which use axial structural members or helical coils to support the in-line stent framework. In principle, the use of a multi-segmented stent may be viewed as less harmful to the tissue of the blood vessel wall, due to a significant increase in the axial flexibility of the stent when compared against conventional devices, which have significant compliance mismatch existing at the junction between the stent ends and the host arterial wall. In addition, the spacing of the individual segments can be adjusted during deployment to achieve customizable vessel wall coverage and customizable radial force. This enables the user to apply more (for example, when treating soft thrombotic lesions) or less (for example, when treating hard calcified lesions) vessel wall coverage as required. The disclosed invention can operate in a host of different lumen shapes and sizes, thereby superseding the functionality of the majority of peripheral vascular stents.

U.S. Pat. No. 6,251,134 describes a stent of high longitudinal flexibility, comprising ring elements commonly aligned along a longitudinal axis of the stent. The ring elements are not physically attached to one another but adjacent rings are coupled via male/female coupling elements, which mate with coupling elements on adjacent rings. U.S. Pat. No. 6,398,803 discloses partially encapsulated spaced-apart ring stents in ePTFE having slits or gaps cut into the material. U.S. Pat. No. 4,106,129 discloses a stented bioprosthetic heart valve comprising a single flexible wire frame stent. The wire frame stent is configured to define a triad of axially projecting circumferentially spaced commissure supports capable of being flexed to provide a limited deformability.

US Patent Publication No. 2008/0208311 relates to a stent comprising a plurality of ring structures having axially extending elements which interleave with axially extending elements on adjacent unconnected rings. European Patent Publication No. 0791341 discloses a stent comprising a wire structure defining a substantially cylindrical wall having three-dimensional closed loops transversely oriented to the generator of the cylinder and fixed to the generator at regular intervals. European Publication No. 0472731 discloses stent comprising bendable resilient ring-like members disposed at both ends of a tube and connected to annular wire members.

International (PCT) Publication No. WO 2008/124114 relates to stent segments comprising different material from at least one of the other stent segments, and where the stent segments are either connected to or unconnected from one another. The individual stent segments generally comprise a zig-zag formation having a multitude of crowns at each end of the stent. U.S. Pat. No. 6,187,034 B1 relates to segmented stents comprising multiple separate segments axially spaced from each other. The segments are joined together with axial elements. The stents also comprise a multitude of crowns at either end.

Despite the plethora of devices that exist, there is a still a need for a device that can withstand significant axial, radial, torsional and bending deformation, or combinations thereof, without fracturing or causing significant injury to the lumen in which it is positioned. Also, there is still a need for the ability to tailor the length and vessel wall coverage of the stent during the surgical procedure while allowing the device to conform to the lumen over a wide range of diameters.

### OBJECT OF THE INVENTION

It is thus an object of the invention to provide a stent which is better suited to peripheral blood vessel applications. A further object is to provide a stent with increased flexibility, customisable length, wall coverage, force and conformance to a wide range of lumen diameters. Further objects of the invention include providing a stent which can reduce vascular injury and reduce or eliminate stent fracture and displacement. It is also an object to provide a stent which can encourage uniform distribution of blood vessel wall shear stress in the stented blood vessel segment as well as downstream from the stented segment. It is also an object to provide a stent which can conform to non-circular sections more easily.

### SUMMARY OF THE INVENTION

According to the present invention there is provided a stent comprising:
at least one individual closed hoop member formed from a resilient material, the hoop, in a two dimensional configuration, having a diametral dimension that exceeds the diameter of the largest blood vessel into which it will be inserted; and
the hoop being resiliently deformable or resiliently deformed so as to occupy a spatial envelope of reduced diameter when inserted in a lumen,
wherein when occupying the spatial envelope of reduced diameter, the closed hoop member has at least two substantially diametrically-opposed generally circumferentially extending regions; and
at least two regions that extend at least in part generally axially relative to the generally circumferentially extending regions,
each of said regions that extend at least in part generally axially being disposed between two of said generally circumferentially extending regions.

The diametral dimension of the hoop member which exceeds the diameter of the blood vessel, may be circular, oval, rectangular, square or any other shape which permits insertion into a blood vessel.

In use a plurality of such individual closed hoop members may by axially arranged within the lumen as required or directed by a medical practitioner. Typically a series of axially arranged hoop members may be inserted into the lumen for best possible results. The series may consist of from 1 to 400 hoop members, the number of hoops used being dependant on the patient and the extent of vessel damage. For example, a series of 1 to 150 hoop members may suit treatment of the iliac arteries, a series of 1 to 200 may suit treatment of the infra-popliteal arteries, and a series of 1 to 400 hoop members may suit treatment of the femoro-popliteal arteries. The axial distance between individual hoop members in a series of axially arranged hoop members is in the range of 0 mm to 20 mm in a compressed state. In other words, the hoop members may overlap or nest one within the other, or be spaced apart from one another. If the hoop members nest, they may partially overlap each other or one hoop member may abut a second hoop member. The overall length of the axially arranged series of hoop members is in the range of from 1 mm to 400 mm in an expanded state. During insertion into the lumen the series of hoop members may be axially arranged offset to each other on opposite sides of a central circumferentially defined plane. The hoop members may be deployed so that a pair of hoop members crosses each other providing a maximum of 4 points of contact with each other.

Each of said regions that extend at least in part generally axially may have at least two opposed portions that extend generally axially with respect to said plane substantially defined by the generally circumferentially extending regions, and a generally circumferentially extending portion connecting said opposed portions.

Each of said at least two regions that extend at least in part generally axially with respect to the plane substantially defined by the generally circumferentially extending regions may extend in the same axial direction.

One of said at least two regions that extend at least in part generally axially with respect to a plane substantially defined by the generally circumferentially extending regions may extend in the opposite axial direction from the other of said at least two regions that extend at least in part generally axially.

In particular embodiments at least one pair of the generally axially opposed regions may be arranged with an axial separation between them. In other words the opposed regions are offset from one another. In such embodiments the hoop member has the appearance of a helix. The helix may have a number of helical turns.

Each of said hoop members may be configured for expansion from a compressed fitting disposition in which said at least two regions that extend at least in part generally axially are at a first transverse spacing from one another to a second expanded disposition of use in which said at least two regions that extend at least in part generally axially are at a second transverse spacing from one another, the second spacing being greater than the first spacing.

The generally circumferentially extending portion may have a geometry selected from the group comprising loop, anfractuous, and crenellated structures.

The hoop profile may have an aspect ratio [length in a diametral dimension:axial length] of between 1:1 and 1:0.5, when occupying a spatial envelope of reduced diameter when inserted in a blood vessel or when expanded in a blood vessel. In certain embodiments the aspect ratio may be 1:1.

The hoop member may have an articulation angle that is selected such that the increase in radial force per unit length delivered onto the lumen equals or exceeds the decrease in radial force per unit length associated with the strain energy within the stent material so that the hoop member shortens axially with radial expansion. Suitably the articulation angle is in the range 60° to 170°. Particularly preferred are articulation angles in the range 60° to 120°. The articulation angle is calculated with the stent ring unwrapped into a two dimensional configuration.

The articulation angle may be defined as the angle between the at least two axially opposed portions (or axial struts) in the region of the generally circumferentially extending portion (or crown) connecting the opposed portions.

Prior art self-expanding stent designs typically have an aspect ratio of 1:0.2 to 1:0.5 for individual stent ring members with a typical articulation angle of 20 to 60°. The individual hoop member of this invention typically has an aspect ratio in the range of 1:0.5 to 1:1 and an articulation angle of greater than 60°.

The hoop members may be nested on a wire, a mandrel or an assembly such as a guide wire. The hoop members may be nested on a guide wire and/or encapsulated by a sheath constraining the hoop members. The hoop members may be balloon expandable.

The resilient material is selected from the group comprising metals, plastics, ceramics or composites thereof. Suitable resilient materials include shape memory alloys such as copper-zinc-aluminum-nickel, copper-aluminum-nickel, iron-manganese-silicon, and nickel-titanium (NiTi) alloys or shape memory polymers manufactured using multi block co-polymers synthesised from MDI/1,4-butanediol, poly(ε-caprolactone), poly(tetrahydrofuran), poly(ethylene adipate), poly(ethylene terephthalate), poly(ethylene oxide), poly(1,4-butadiene), Polyethylene, poly(vinyl acetate), polyamide-6 (nylon-6), or, poly(2-methyl-2-oxazoline) could be utilized. Certain ZrO₂ ceramics could also be utilised for their shape memory effects.

The hoop member may be coated with a biodegradable material selected from the group comprising Poly-L-lactic acid (PLLA), polyglycolic acid (PGA), poly(D,L-lactide/glycolide) copolymer (PDLA), polycaprolactone (PCL), magnesium, or any other biodegradable material. The hoop member may act as a carrier for a drug to be delivered to the blood vessel. The hoop member may be coated with a bioabsorbable drug eluting material, or contain a drug within it. The hoop member may also be formed from a biodegradable material selected from the group comprising Poly-L-lactic acid (PLLA), polyglycolic acid (PGA), poly(D,L-lactide/glycolide) copolymer (PDLA), polycaprolactone (PCL), magnesium, or any other biodegradable material.

In another aspect, each hoop member may be shaped to have an initial relaxed disposition from which it is resiliently deformable into a support configuration in which the hoop member occupies a lesser spatial envelope than it occupies in its relaxed disposition while retaining an expanding bias into supporting engagement against the internal wall of the lumen into which it has been inserted, and the hoop member is further resiliently deformable into an insertion configuration in which the hoop member is constrained to occupy a still lesser spatial envelope than that which it occupies in the support configuration, so that the hoop can be inserted into the blood vessel while retaining an expanding bias towards the support configuration, and the hoop member is shaped to have a plurality of opposed hoop portions, the spacing of which in a direction transverse to the axial direction of the stent is reduced, in the insertion configuration, relative to the spacing of these portions in the support configuration, by spring-clip-like resilient deformation of the hoop member, and each of the opposed hoop portions has a substantially curving profile in the direction of extent of the material of the hoop member.

The opposed hoop portions may be interconnected by hoop member portions which provide at least in part the resilient bias against which resilient deformation of the hoop member from the insertion configuration towards the support configuration may be effected.

When the device is compressed for insertion into the blood vessel, most of the resilience or springiness may be provided by the flexing of the interconnecting legs or portions which extend between the curving or arcuate opposed hoop portions, with the curving portions remaining relatively unchanged in shape. Thus there can be quite significant compression with the curvature of the opposed portions increasing only very slightly.

The hoop member may be symmetrical and has an axial dimension in both its support configuration and its insertion configuration, each of the opposed hoop portions defining a reversal in the direction of extent of the material of the hoop member relative to the axial direction of the stent, and there are at least two opposed hoop portions.

The hoop member portions interconnecting the opposed hoop portions may themselves each define one of a pair of further hoop portions. The further hoop portions may themselves also be mutually opposed.

Each further hoop portion may be located to an opposite side of the opposed hoop member portions relative to the axial direction of the stent.

The stent may comprise two opposed hoop portions and two further hoop portions.

In still another aspect of the invention, the hoop member can be considered to have a saddle shape in the compressed configuration, with four elongate elements connected by generally curved elements. The curved elements or apexes or crowns are substantially U-shaped, C-shaped or V-shaped. Each hoop member may have two crowns at the proximal end and two crowns at the distal end of the stent.

A reduced number of crowns in the hoop member facilitates a reduced crimp diameter in the collapsed configuration; therefore the stent can be crimped into a smaller diameter delivery system allowing smaller lumens and vessels to be accessed. Alternatively the crown radius can be increased to give a better distribution of strain energy for a fixed crimp diameter. It also allows for an increased strut width and/or increased overlapping of hoop members per unit length for the same diameter of delivery system.

Thus in one embodiment with four elongate elements the end view of the stent has a rectangular profile which is compressed down to a circular profile. This is achieved by forming the geometry over a non-circular profile in three dimensions. However, in embodiments with more than four elongate elements, the end view profile would not be rectangular. For example with six elongate elements, the profile would be hexagonal, and with eight elongate elements the profile would be octagonal.

The hoop member may be provided with an anchoring means which is adapted to anchor the stent against the lumen wall and reduce or eliminate movement of the stent when in situ. Each of the generally circumferentially extending regions, generally curved elements or apexes or crowns may be tapered, which increases pressure locally on the lumen so as to give a better grip and to anchor the stent in place. Also, the outer surface of the hoop member may have a surface finish or profile so as to increase the surface area in contact with the lumen, hence giving a better grip to anchor the stent in place.

The individual closed circular hoop members in a multiple hoop member configuration may be of alternating hoop diameters.

The invention also provides a stent as defined above together with compression means for constraining at least one hoop member in the compressed fitting disposition for placement of the stent within a lumen, the compression means being subsequently removable to allow the hoop member to expand individually, to a respective second expanded disposition in which axial and circumferential regions of the hoop member engage against the inner wall of the blood vessel.

A multi-segmented stent according to the invention comprising super-elastic Nitinol in the form of a series of unconnected reinforcing “hoops” radically reduces the axial and torsional stiffness of the stented blood vessel but retains the radial stiffness required to reinforce the lumen of that artery. For certain embodiments, each segment, no matter what orientation it takes within the lumen, expands radially to allow flow through with minimal disturbance and yet prevents the lumen wall from collapsing.

The clinician may no longer be limited to the overall length of the stent to be deployed, as multiple segments can be introduced to whatever overall length the clinician desires.

A 3D aspect ratio close to 1:1 enables deployment in any plane without significant flow obstruction.

Multiple deployment of the basic hoop profile provides customisable blood vessel wall coverage; tighter spacing provides greater wall coverage.

Axial stiffness of the stented blood vessel is radically reduced but the radial stiffness required to reinforce the lumen is retained.

A multi-segmented stent has a distinct advantage over current practice, due to the significant increase in the axial, torsional, and bending flexibility of the stent in comparison to conventional designs. This increase in flexibility may reduce vascular injury (hence reducing restenosis), improve the haemodynamic flow within the blood vessel (hence reducing thrombus formation), and reduce or eliminate stent migration, stent strut fractures, and vessel rupture and dissections. In addition, such a flexible multi-segmented stent is capable of being delivered more easily into highly torturous peripheral blood vessels. Additionally, for some embodiments, subsequent rotation of the stent will not block arterial flow after deployment.

The geometry of the hoop members when positioned in a lumen induces a uniform distribution of blood vessel wall shear stress, without significantly distorting the lumen central axis.

The invention also provides a kit of parts comprising at least one hoop member as defined above mounted within a sheath, or on a wire, mandrel or guide wire. The kit may comprise a plurality of hoop members mounted along the sheath or guide wire at varying axial spacings, or axially off-set with respect to one another or at varying angular spacing when viewed in the axial direction such that they can be deployed at varying spacings or in varying orientations within a blood vessel.

### BRIEF DESCRIPTION OF THE DRAWINGS

Additional features and advantages of the present invention are described in, and will be apparent from, the detailed description of the invention and from the drawings in which:

FIG. 1A illustrates a perspective, elevation, plan, and end view of the geometry of a first of the embodiments of a single closed circular hoop member of an intravascular stent of the present invention;

FIG. 1B illustrates a perspective view of an alternative embodiment or variant of a single closed circular hoop member according to the invention to be deployed in a blood vessel, body lumen, or duct.

FIG. 2 illustrates a perspective, elevation, plan, and end view of the hoop member of FIG. 1A when deployed in a multiple hoop member configuration.

FIG. 3 illustrates an elevation, plan, and end view of the geometry of another embodiment of a single closed circular hoop member of an intravascular stent of the present invention.

FIG. 4 illustrates an elevation, plan, and end view of the hoop member of FIG. 3 when deployed in a multiple hoop member configuration.

FIG. 5 illustrates an elevation, end, and perspective view of the geometry of an offset multiple closed circular hoop member configuration of an intravascular stent of the present invention.

FIG. 6A illustrates an elevation, end, and plan view of one of the geometry of one ring or hoop of a helix forming embodiment of the closed circular hoop member of an intravascular stent of the present invention;

FIG. 6B illustrates an elevation, end, and isometric view of closed circular hoop members of FIG. 6A when deployed offset from each other in a multiple closed circular hoop member configuration.

FIG. 7 illustrates an isometric view of an embodiment of an intravascular stent of the present invention wherein the individual closed circular hoop members are arranged to deploy across or over each other.

FIG. 8 illustrates an example of a closed circular hoop member for which, when deployed in a blood vessel, the geometry of the hoop member changes from the geometry of an unformed hoop to any one of the geometries illustrated in FIGS. 1 to 7.

FIG. 9 is a perspective view of an embodiment of a single closed circular hoop member of an intravascular stent of the present invention formed from a wire whose ends are joined and which has an anchoring means at the crowns.

FIG. 10a is a perspective view of the hoop member with arrows showing the positions at which force is applied to compress the member for nesting on a wire or a guide wire.

FIG. 10b is a diagrammatic representation of the hoop member of the stent when nested on a guide wire for insertion into a lumen.

FIGS. 11a, b, and c are a diagrammatic representation of a stent of the invention being delivered to a site within a blood vessel by means of withdrawing the constraining wire and/or sheath.

FIG. 12A illustrates a perspective view of the geometry of a single closed hoop member with at least one pair of generally axially opposed regions of an intravascular stent of the present invention.

FIG. 12B illustrates an elevation, end, and perspective view of the geometry of a single closed hoop member with at least one pair of generally axially opposed regions of an intravascular stent of the present invention;

FIG. 12C illustrates an elevation, end and perspective view of the geometry of a single closed hoop member with at least two pairs of generally axially opposed regions arranged with an axial separation between them.

FIG. 13A illustrates an elevation view of an intravascular stent of the present invention, demonstrating the articulation angle thereof.

FIG. 13B illustrates the fixed perpendicular separation and circumferential distance of a pair of closed hoop members an intravascular stent of the present invention, demonstrating the articulation angle thereof.

FIG. 13C illustrates an isometric view of a group of closed hoop members illustrated in FIGS. 13A and 13B when deployed in a multiple hoop member configuration.

FIG. 14 illustrates the change in axial length of a closed hoop member of an intravascular stent of the present invention in situ in a vessel when the vessel is under axial tension and reduces in diameter.

FIG. 15 illustrates (an end and isometric view of a closed hoop member of an intravascular stent of the present invention) that where the hoop member is subject to a point or crush load, the hoop member exerts a minimal or reduced resultant force or deformation in a perpendicular direction to the load.

FIG. 16A illustrates an elevation, and perspective view of the geometry of another embodiment of a single closed circular hoop member of an intravascular stent of the present invention.

FIG. 16B illustrates an elevation, plan and perspective view of the hoop member of FIG. 16A when deployed in a multiple hoop member configuration and in a collapsed configuration.

FIG. 17A illustrates an elevation, plan, end, and isometric view of the geometry of another embodiment of a single closed circular hoop member of an intravascular stent of the present invention where the proximal crowns have an angular offset relative to the distal crowns.

FIG. 17B illustrates an end, elevation, and plan and perspective view of the geometry of another embodiment of a single closed circular hoop member of an intravascular stent of the present invention where the crowns are axially offset on the proximal end or distal end.

FIG. 18 illustrates an elevation, plan, end, and perspective view of the geometry of another embodiment of a single closed circular hoop member of an intravascular stent of the present invention.

FIG. 19A illustrates a perspective view of the geometry of another embodiment of a single closed circular hoop member of an intravascular stent of the present invention, while FIG. 19B illustrates a variation of the geometry of FIG. 19A.

FIGS. 20A and 20B illustrate a perspective view of the geometries of another embodiment of a single closed circular hoop member of an intravascular stent of the present invention.

FIG. 21 illustrates an elevation, end, and perspective view of the geometry of another embodiment of a single closed hoop member of an intravascular stent of the present invention where the end view is oval.

FIG. 22 illustrates an elevation, end, and perspective view of the geometry of another embodiment of multiple closed circular hoop members of the intravascular stent of FIG. 21 showing a radial off-set.

FIG. 23 illustrates an elevation, plan, end, and perspective view of the geometry of another embodiment of a single closed hoop member of an intravascular stent of the present invention.

FIG. 24 illustrates a perspective view of a number of closed hoop members of an intravascular stent of the present invention in a collapsed state for loading into a delivery system prior to insertion into the vasculature.

FIG. 25 illustrates an elevation and perspective view of a group of closed hoop members an intravascular stent of the present invention with variable spacing when deployed in a multiple hoop member configuration.

FIG. 26 illustrates an elevation and perspective view of a group of closed hoop members of an intravascular stent of the present invention with modified axial spacing and rotation when deployed in a multiple hoop member configuration.

FIG. 27A illustrates views in planes X, Y, and Z, and a perspective view thereof of a single closed hoop member of an intravascular stent of the present invention in situ in a vessel where fluid flows in more than one axial direction.

FIG. 27B illustrates a plan and perspective view of an example of a single hoop member which can be deployed in a vessel where fluid flows in more than one axial direction.

FIGS. 28A and 28B illustrate a plan and perspective view, respectively, of a group of closed hoop members of varying geometries deployed in a multiple hoop member configuration of an intravascular stent of the present invention in situ in a bifurcated vessel.

FIGS. 29A and 29B show a prior art stent and the stent of the present invention respectively, in a two dimensional configuration, illustrating the articulation angle.

### DETAILED DESCRIPTION OF THE DRAWINGS

It will be readily apparent to one of ordinary skill in the art that the examples disclosed herein below represent generalised examples only, and that other arrangements and methods capable of reproducing the invention are possible and are embraced by the present invention.

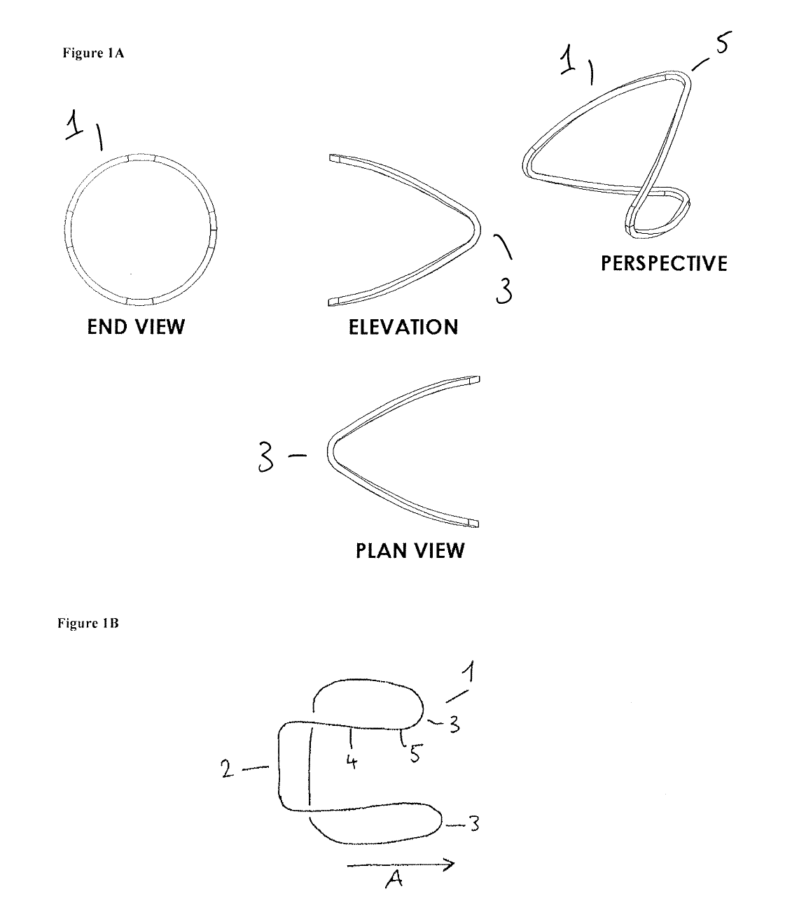

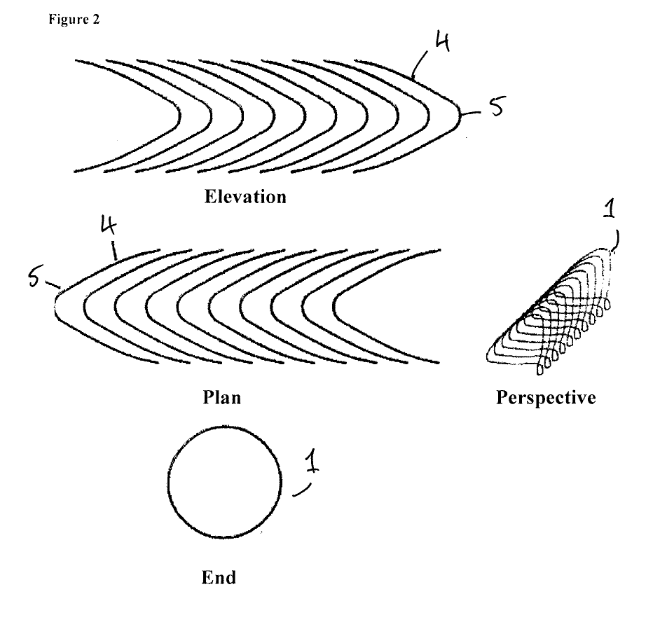

In FIGS. 1A, 1B, and 2 there is illustrated an elevation view, a plan view, an end view, and a perspective view of a single hoop member and multiple hoop members of a stent according to the present invention. The stent is for use within the vasculature or lumens of the body. The hoop member is generally indicated by the reference numeral 1. As with all embodiments of the present invention, the hoop member 1 is a closed circular hoop having a diametral dimension that exceeds the lumen diameter into which it will be inserted. The hoop member 1 is biased towards its fully expanded configuration so that when placed in a vessel of less diameter, the hoop member 1 will move from a compressed configuration towards a more expanded configuration, as illustrated, for example, in FIGS. 11a to 11c.

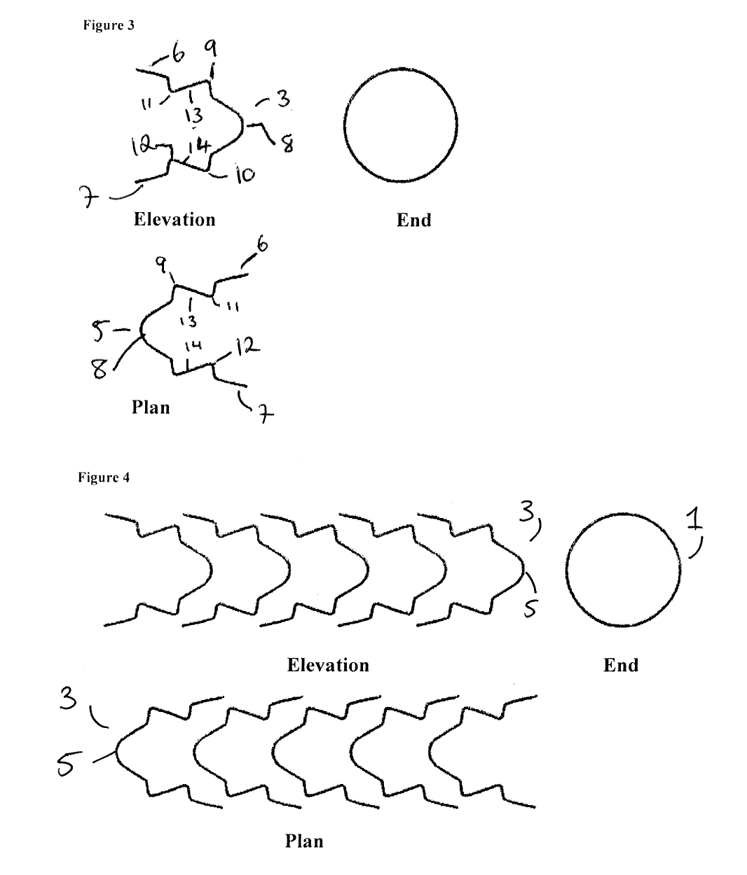

When deployed in a blood vessel, the hoop member 1 as illustrated in FIG. 1B is essentially an annular segment comprising at least two diametrically-opposed generally circumferentially-extending regions 2 and at least two regions 3 that extend at least in part generally axially with respect to a plane substantially defined by the regions 2. The regions 2 may vary geometries, which are clearly illustrated in FIGS. 3, 4, and 16A and 16B and will be discussed later.

In the present embodiment, the regions 2 comprise at least two axially opposed portions (or axial struts) 4 that extend generally axially with respect to the plane substantially defined by regions 2, and a generally circumferentially extending portion (or crown) 5 connecting the opposed portions 4. The two regions 3 extend in the same axial direction, as defined by the arrow A in FIG. 1B, and according to one desired aspect of the invention, one of the two regions 4 extends in the opposite axial direction from the other of the two regions 4. The hoop member 1 when expanded in a blood vessel as depicted in FIG. 1B can have an aspect ratio of 1:1, i.e. the hoop member 1 has the same overall dimension in all three planar directions.

FIG. 2 illustrates the spatial arrangement of a series of multiple hoop members 1 deployed in a nested configuration. The opposed portions 4 and circumferentially extending portions (or crowns) 5 are clearly marked. The nesting distance or the axial distance between the individual hoop members 1 following to deployment in a blood vessel will be in the range of between 0 mm to 20 mm, preferably in the range of 0 mm to 10 mm, and more preferably in the range of 2 mm to 5 mm. It will, however, be appreciated that individual hoop members may be positioned within the lumen so that they are not nested. When the series of multiple hoop members 1 are deployed in a blood vessel, the overall axial length of the stent comprising the series of multiple hoop members 1 will be in the range of between 1 mm to 400 mm, preferably in the range of 10 mm to 400 mm, and more preferably in the range of 30 mm to 300 mm.

FIGS. 3 and 4 show the region 3 of the hoop member 1 having a variety of anfractuous or convoluted structures. In FIGS. 3 and 4, the region 3 comprises axial struts 6,7, a curved outer end 8, and bulges 9,10,11,12 in each of axial struts 6,7. Axial struts 6,7 are joined to opposing portions 4 connecting the two regions 3 (not shown). Abutments 13,14 are located axially along struts 6,7 and between bulges 9,11 and 10,12, respectively. The geometry of region 3 as described above and illustrated in FIG. 3 maintains the 1:1 aspect ratio while increasing blood vessel wall coverage and minimizing flow obstruction if deployed out of plane. When deployed in a blood vessel as a multiple hoop member series, less hoop members 1 may be required to achieve the target length, than when region 3 is a loop structure, due to the increased blood vessel wall coverage provided by the geometry of the region 3.

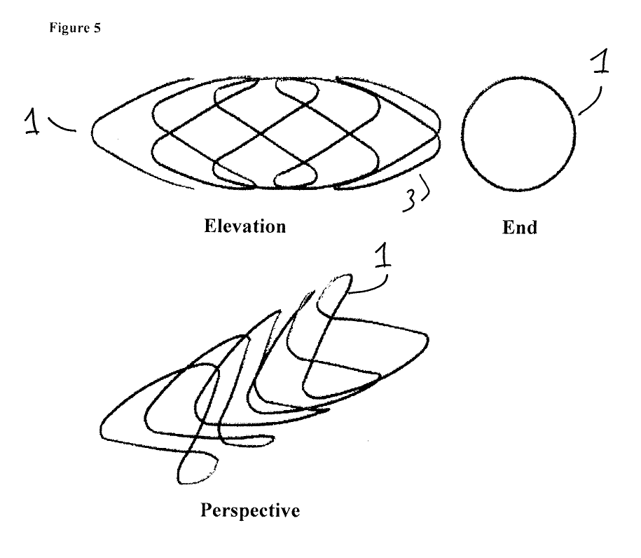

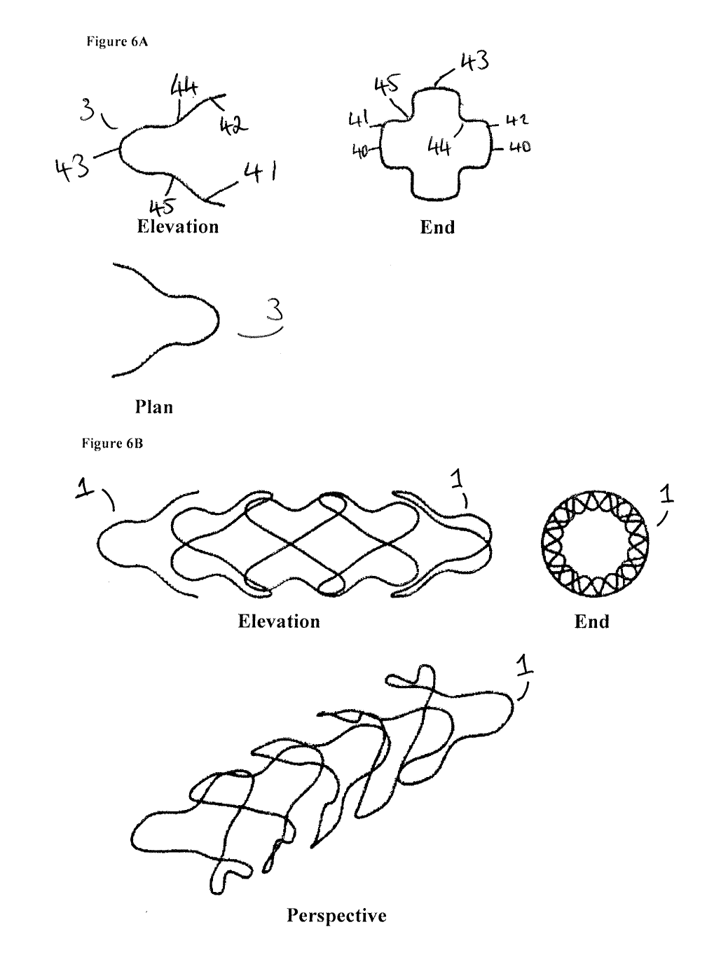

FIG. 5 shows the hoop member 1 as described in FIG. 1 having an offset orientation where the axial regions 3 are at an angle between 1 degrees to 180 degrees, preferably between 1 degrees to 90 degrees, and more preferably between 1 degrees and 45 degrees to the blood vessel axis. The variation in orientation of the region 3 in relation to the blood vessel axis provides a helical pattern varying the blood vessel stiffness along the stented segment, which can encourage uniform distribution of wall shear stress.

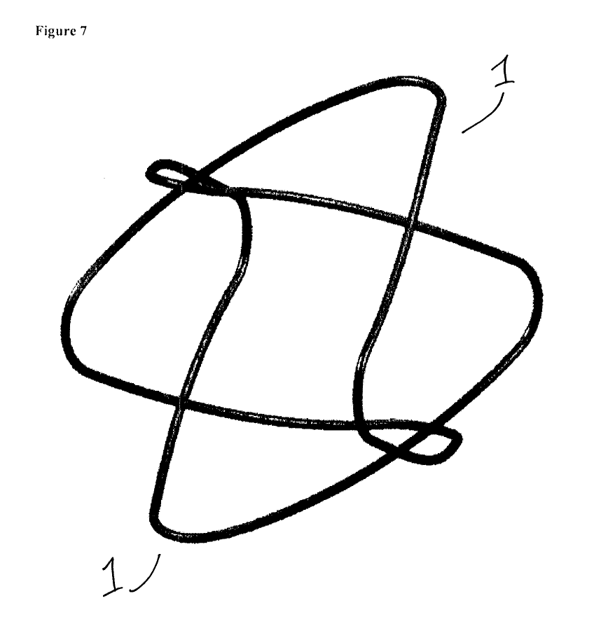

FIG. 6A shows the region 3 in this embodiment comprising axial struts 41,42, a curved outer end 43, and concave abutments 44,45. The concave abutments 44,45 joining the curved outer end 43 with axial struts 41,42 form a crenellated structure when viewed end on. In FIG. 6B, the geometry of hoop member 1 is utilised to remodel the blood vessel into a non-circular profile. The individual hoop members 1 are deployed offset to each other in a helical pattern intended to induce uniform distribution of wall shear stress.

By varying the diametral dimensions of the hoop members 1, a series of multiple hoop members having alternating diameters may be deployed to facilitate tapering or stepped blood vessel lumens and further facilitate a variable axial stiffness of a stented blood vessel.

FIG. 7 shows the hoop member 1 in an interfering structural dimension. A pair of hoop members 1, depicted here with the geometry of FIG. 1, may be deployed across or over each other, resulting in a maximum of four contact points with each other.

The use of multi-segmented stents comprising a single or series of hoop members 1 of the present invention radically reduces the axial stiffness of the stented blood vessel, yet retains radial stiffness required to reinforce the blood vessel lumen. The hoop member 1 of the present invention allows a three-dimensional self-orientation in a blood vessel. The shapes and geometry of the three-dimensional hoop member 1 ensure maximal flexibility in all directions. Consequently, the hoop member 1 will respond with minimum resistance to torsion, bending and axial loading, while maintaining optimum radial strength properties against compression loading. Furthermore, in certain embodiments, the flow in any direction, independent of the stent segment final orientation at equilibrium, is unobstructed.

This invention increases the radial force of a stent ring by reducing the number of circumferentially extending portions (or crowns) 5, preferably to two crowns at the proximal and distal end of the hoop member. In this configuration, when viewed in two perpendicular planes such as a plan and elevation view, the stent ring appears to have a generally ‘C’ or ‘V’ shape (for example, as shown in FIG. 1).

The axial length of the stent ring can be maintained by increasing the strut length so that the aspect ratio also remains unchanged. The aspect ratio is defined to be the ratio of the length in a diametral dimension to the axial length. Maintaining or increasing the aspect ratio gives the stent ring stability during the expansion process and also when fully expanded within the vessel. It can also minimise the likelihood of migration in the vessel after deployment.

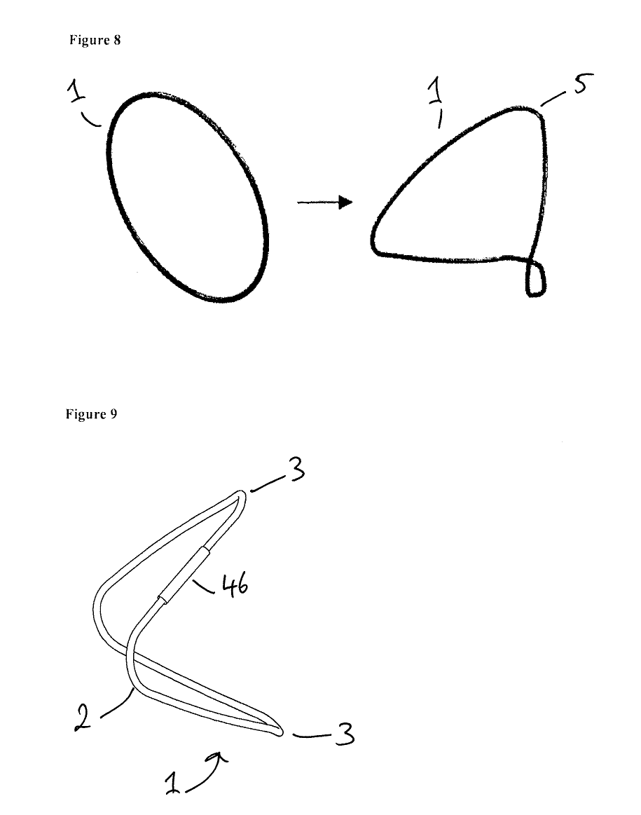

The closed hoop member 1 of the present invention is manufactured by methods commonly known in the art. For example, the hoop member 1 can be manufactured from a continuous loop shaped into a three-dimensional configuration with an aspect ratio approximating 1:1 by setting the hoop around a cylindrical tube with the desired inner diameter. Alternatively, the hoop member 1 may be manufactured from round wire cut to length, joining the wire ends (for example by laser welding) and electro-polishing for a smooth joint. This may then be heat formed into a particular profile or positioned on a mandrel so that upon deployment, it assumes the desired hoop member configuration. For example, use of a four sided mandrel results in a rectangular profile. Another alternate manufacturing method comprises laser-cutting the hoop member 1 from a tube of resilient material (for example, Nitinol (nickel/titanium alloy)) of required thickness as an annular hoop or as a three-dimensional shaped hoop approximating that of the desired three-dimensional shape when the hoop member 1 is in an expanded shape. Alternatively, the hoop shape may be formed through cutting or stamping a profile from a 2 dimensional sheet of material and subsequently shaped into the desired three-dimensional shape. Also, the hoop shape may be formed from a tubular material where the ends are joined by placing one end inside another and/or through the use of a mandrel inside the tubular material. Finally, the hoop member may be wrapped around a forming mandrel and subsequently heat formed before being assembled via a connecting radiopaque marker and to aid fluoroscopic visualisation. FIG. 8 demonstrates that a hoop member 1 with region 3 having a geometry similar to that of any of the previously described geometries is formed during the process of loading the unformed hoop member 1 into a delivery catheter or directly in a lumen. When the hoop member is deployed, the geometry of region 3 may resemble any of the previously described region 3 geometries depending on the forming process utilised.

FIG. 9 shows an alternative embodiment of the hoop member in its compressed state. In this case the hoop member is formed from a wire, the ends of which are held together by a cylindrical fixing means 46. This embodiment is provided with an anchoring means at the crown, in the form of a lip 46a, which bears against the vessel wall thus holding the loop member in place.

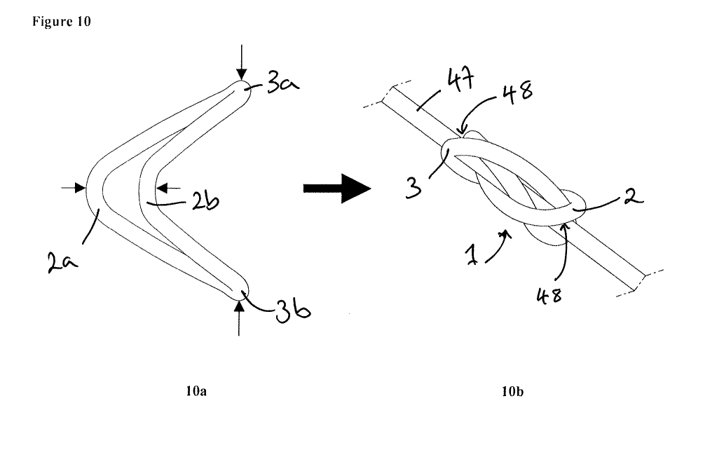

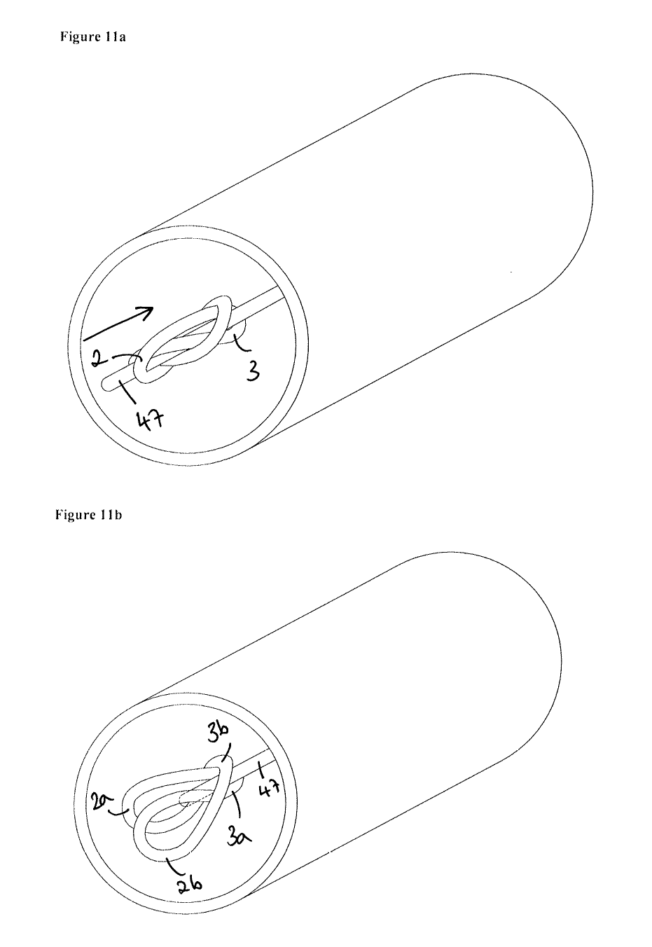

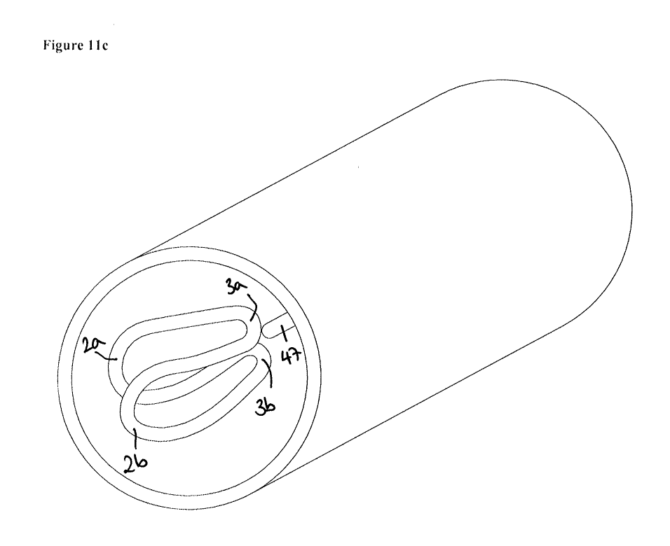

The hoop member of the invention may be constrained for nesting on a wire, mandrel or guide wire, as shown in FIGS. 10a and 10b. To nest the hoop member, force is applied to each of the regions 2 and 3 in the direction of the arrows marked on the figure. The application of force pushes portion 3a towards the corresponding portion 3b, and pushes portion 2a towards portion 2b. By pushing further, portion 3a moves through portion 3b to create an aperture 48 between them. A similar aperture is created between 2a and 2b. It is then possible to insert a wire, a mandrel or guide wire 47 into these apertures as shown in FIG. 10b. In this way it is possible to insert a series of hoop members onto a guide wire for insertion into a lumen.

FIGS. 11a, b and c show a diagrammatic representation of the stent of the invention being fitted within a blood vessel lumen. As the stent is released from the wire, guide wire and/or sheath portions 2a and 2b move apart and towards the vessel wall. Further retraction of the wire, guide wire and/or sheath releases portions 3a and 3b, which also move apart and into contact with the vessel wall.

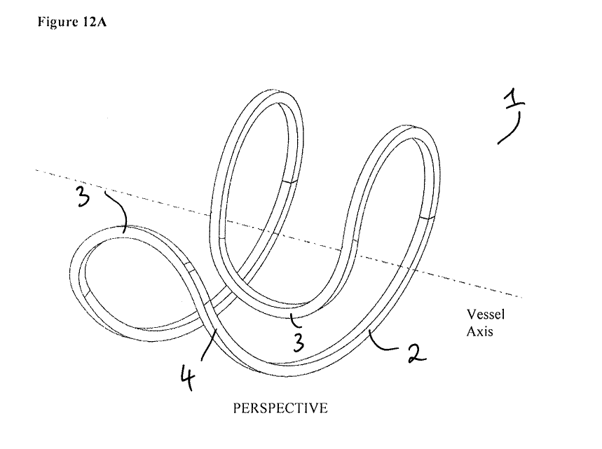

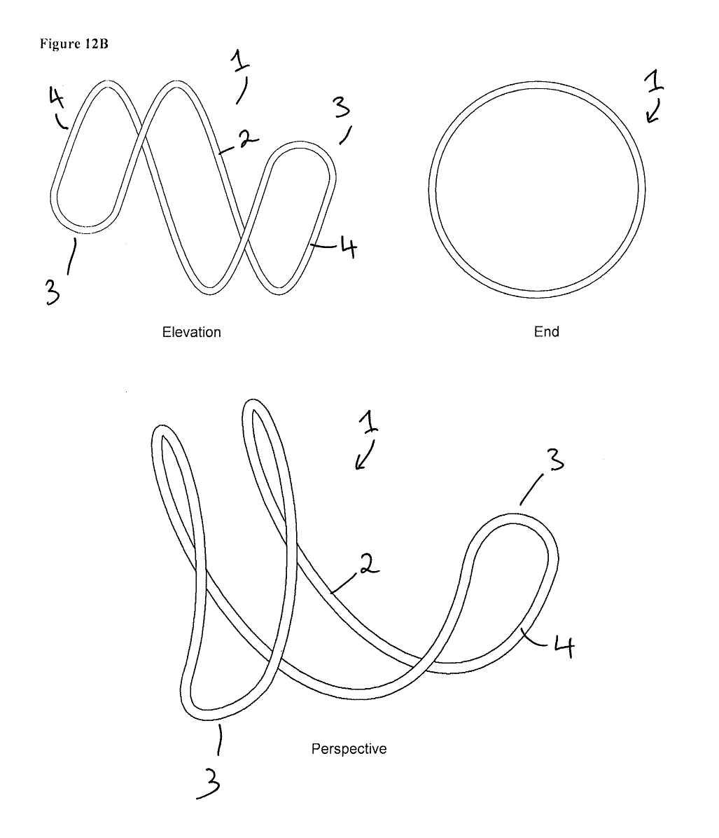

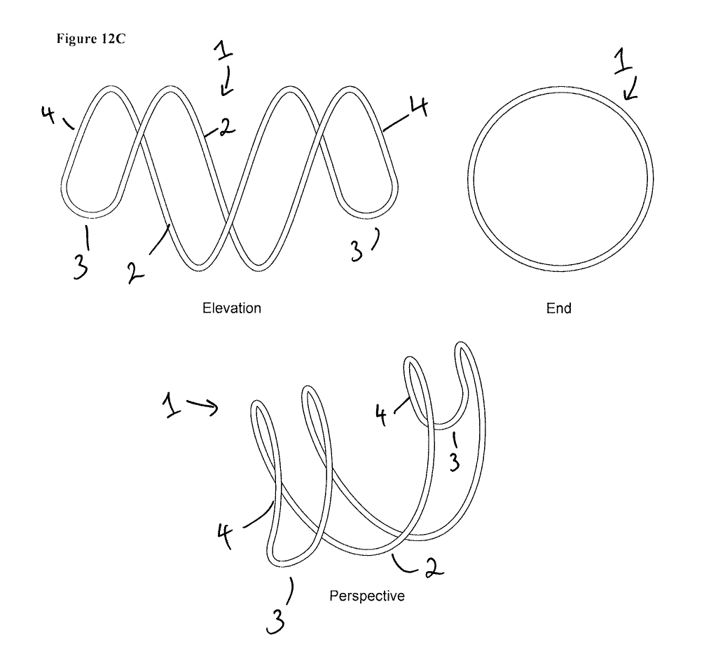

In FIGS. 12A, 12B and 12C there is illustrated an elevation, end, and perspective view of the geometry of an alternative embodiment of the single closed hoop member as described in FIG. 1. The single closed hoop member has at least one pair of generally axially opposed regions 3 that extend at least in part generally axially with respect to a plane substantially defined by the regions 2, arranged with an axial separation between them, forming an offset coiled hoop. This geometry may provide a means of increasing the wall coverage of the stent, and may also allow safer delivery of the hoop segments. Additionally, this embodiment may allow improved flow within the lumen following tissue response, such as inflammation or restenosis, occurring due to such regions, 2, being offset axially from each other. The lumen may therefore allow increase flow which may result in uniform distribution of wall shear stress.

The hoop member 1 may be composed of resilient material such as metals, plastics, ceramics or composites thereof. The super-elastic and shape-memory properties of the resilient material used to manufacture the self-expanding hoop members 1 will ensure continuous engagement of the stent against the blood vessel wall. The hoop member will expand in the blood vessel until equilibrium between the stent and the blood vessel wall is established, thereby controlling the stent diameter in situ.

Another aspect of the present invention is to manufacture the hoop member 1 from a biodegradable material or coating the hoop member 1 with a biodegradable material that will act as a carrier for a drug to be delivered to the stented blood vessel. A biodegradable material can be any known to those skilled in the art, for example: Poly-L-lactic acid (PLLA), polyglycolic acid (PGA), poly(D,L-lactide/glycolide) copolymer (PDLA), polycaprolactone (PCL), and magnesium, or any other biodegradable material.

The hoop member 1 may be deployed using any of the means known to those skilled in the art. The self-orientation characteristic of the hoop member 1 results in a reduced requirement for control during stent delivery. For example, the hoop member 1 or a series of hoop members 1 may be delivered to a blood vessel by nesting or constraining the hoop members 1 on a wire, mandrel or guide wire into the delivery configuration and released into the blood vessel by retracting the wire against a stopper on a catheter tip. Alternatively, the hoop members can be contained within a delivery sheath over a wire, mandrel or guide wire and deployed by retracting the sheath against a guide wire lumen stop. Another desired means is encapsulating the individual hoop or series of hoop members 1 in a biodegradable sheath that also can be used as a drug delivery interface between the hoop members and the blood vessel. The sheath material can be designed to be stiff in dry conditions and elastically deformable when in contact with fluid in the blood vessel at body temperature.

Another desired aspect of the present invention is to deliver the hoop members 1 nested on a guide wire/tube and encapsulated by a sheath constraining or compressing the hoop members 1 therein. Maintaining the guide wire/tube in a fixed position and retracting the sheath delivers the hoop members 1 to the blood vessel.

A further desired aspect of the present invention is the delivery of the nested hoop members 1 by means of an expandable balloon. The nested hoops can be arranged in a linear or helix arrangement and expanded by inflating the balloon.

Flattened hoop members 1 can be delivered in the shape of a wire pre-set to the desired shape, using the resilient material having elastic properties, which does not require a guide wire on which to nest the hoop members 1. The flattened hoop members may also be delivered encapsulated by a sheath.

A further desired aspect of the present invention, which is an improvement over the existing delivery catheters known in the art, is to introduce an additional torque angle control in order to induce a helix effect to any stent segments by either turning the catheter or providing an additional twist to the delivery catheter. A slight rotation of the delivery device, coupled with the natural anatomical variation of the diseased artery segment can result in the hoop members 1 being axially arranged off-set to each other on opposite sides of a central circumferentially defined plane in a helical arrangement. This helical hoop arrangement will induce a uniform distribution of wall shear stress in the stented blood vessel segment as well as downstream from the stented segment.

A further embodiment of the present invention is to coat the hoop member with ePTFE, Polyamide, Polyimide, PET, or a similar type of biocompatible material.

The multi-segmented stent has a distinct advantage over current practice due to the significant increase in the flexibility of the stent in comparison to conventional designs. This increase in flexibility may reduce vascular injury (hence reducing restenosis) improve the haemodynamic flow within the blood vessel (hence reducing thrombus formation), and reduce/eliminate stent migration, stent strut fractures, vessel dissection, and vessel rupture. Furthermore, due to the delivery flexibility of the hoop member 1, the hoop members 1 can be deployed in very small vascular lumens, i.e. for example in neurosurgical, coronary, below-knee applications and the like.

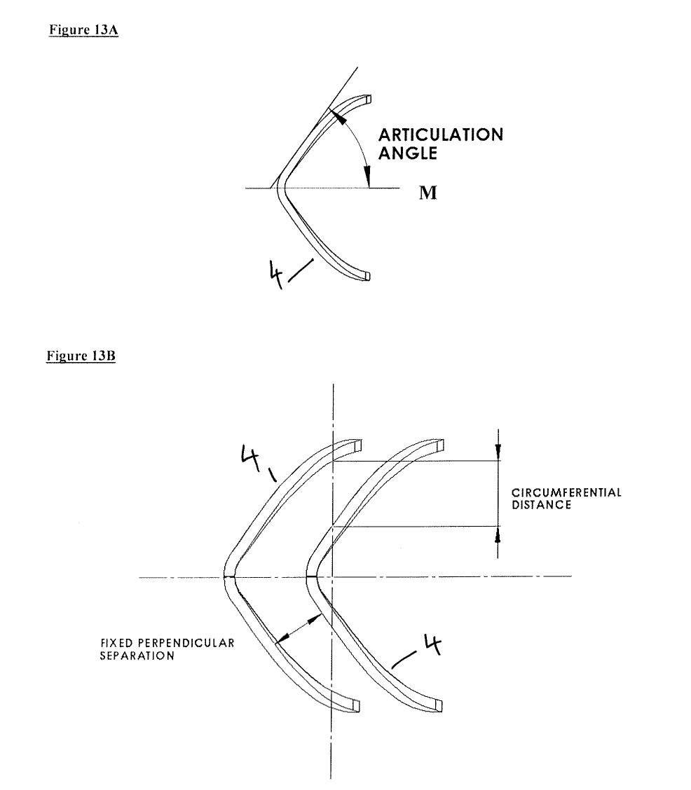

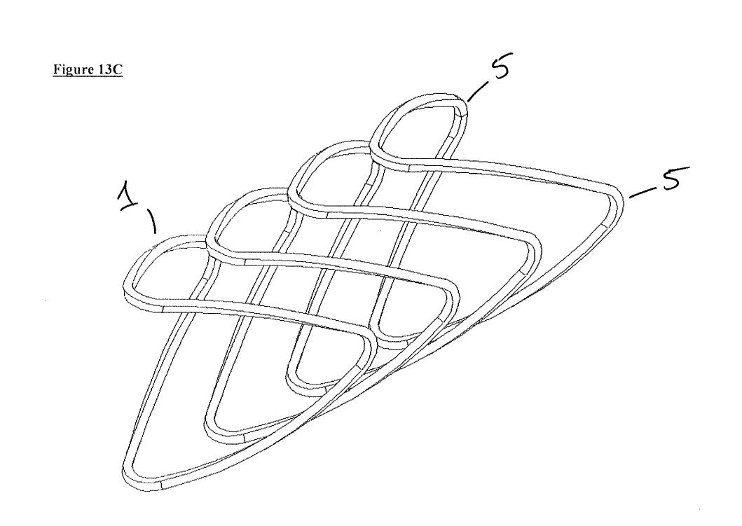

FIG. 13A to 13C illustrate a plan (A and B) and perspective (C) view of the hoop member 1 of the present invention. The hoop member 1 in plan (and elevation) view has a “C” or “V” shape and emphasises the articulation angle formed by the opposed portions 4 from an axis M. In this configuration, the hoop member 1 has a minimised number of generally circumferentially extending portions (or crowns) 5 and the articulation angle is increased to give a higher radial force per unit length of stent (FIG. 13A). This causes the aspect ratio to decrease, but due to the multi segmented nature of this stent, the foreshortening between the proximal and distal ends of the entire stent is negligible as the foreshortening predominantly affects the gaps between the individual hoop members. However this gap and in addition the vessel wall coverage can be controlled by varying the number of hoop members deployed per unit length. By varying the number of hoop members deployed per unit length, adequate vessel wall coverage can be maintained, foreshortening is minimised, while the significant benefits of increased radial force, and increased axial and torsional flexibility are achieved (see for example, FIG. 13C).

The stent comprising a series of hoop members of the present invention provides for an improved flexural and radial stiffness transition from the unstented section of the host artery to the stented artery. This is a benefit as intimal hyperplasia can occur at the ends of stents where there is a sudden bending and radial stiffness transition from the stented portion of the artery to the unstented section. This hyperplasia may be partially attributed to vessel trauma, which can occur due to the sudden change in stiffness and relative movement of the stented and unstented artery sections as a result of vessel mobility.

The improved flexural and radial stiffness transition occurs in the hoop members of the stent of the present invention because the number of circumferentially extending portions (or crowns) 5 is reduced when compared to stents of the prior art, hence the number of contact points between the stent and the vessel wall is minimised at the proximal and distal ends and increases at sections in between. Therefore the flexural stiffness of the entire stent or individual hoop member is not consistent along the vessel axis but decreases as one approaches the proximal and distal ends.

The reduced flexural and radial stiffness transition is achieved by having a lower number of contact points at a proximal or distal section of the stent than at a section in the mid region. This characteristic also gives the benefit of having a transition in vessel wall coverage as the number and distribution of the stent contact points change as you approach the proximal and distal ends.

In the stent of the present invention, the articulation angle increases as the number of circumferentially extending portions (or crowns) 5 decreases. As this angle increases, the circumferential distance between struts also increases for a fixed perpendicular separation between the hoop members. This increased circumferential separation reduces the torsional stiffness of the stented vessel (see FIG. 13B). As stated above, for a reduced number of circumferentially extending portions (or crowns) 5, the articulation angle increases. As this angle increases the proportion of stent material aligned in the axial direction is reduced hence increasing axial compliance.

In addition when the vessel is under axial tension and reduces in diameter (as illustrated in FIG. 14, for example), the hoop member 1 increases in length axially, facilitating the expansion of the vessel in the axial direction (arrow F), making it more axially compliant in tension and compression.

As the hoop member 1 of the present invention has a reduced number of circumferentially extended portions (or crowns) 5 and increased strut articulation angles, a single size hoop member 1 may be used to treat a broad range of indicated vessel diameters. This is due to the hoop member 1 providing a usable radial force across a bigger range of indicated vessel diameters as a stent comprising a number of the hoop members 1 expand from a crimped configuration. As the stent expands the radial force does not decrease but can remain constant as the expanded diameter increases. In some cases the radial force can increase as the expanded stent diameter increases.

This occurs due to the large articulation angles of the struts of the hoop member 1. For a unit change in expanded diameter, the change in articulation angle is small for each circumferentially extended portion (or crown) 5 in a stent ring with multiple crowns. However in a stent ring with a low number of crowns 5 proximally and distally, for example two crowns, the change in expanded diameter causes a larger increase in articulation angle. Therefore there is a greater increase in the radial component of the force in the hoop member 1 with the reduced number of crowns. As the stent expands, the radial component of the force balances or exceeds the decrease in strain energy in the crown, allowing the total radial force per unit length to remain constant or even increase.

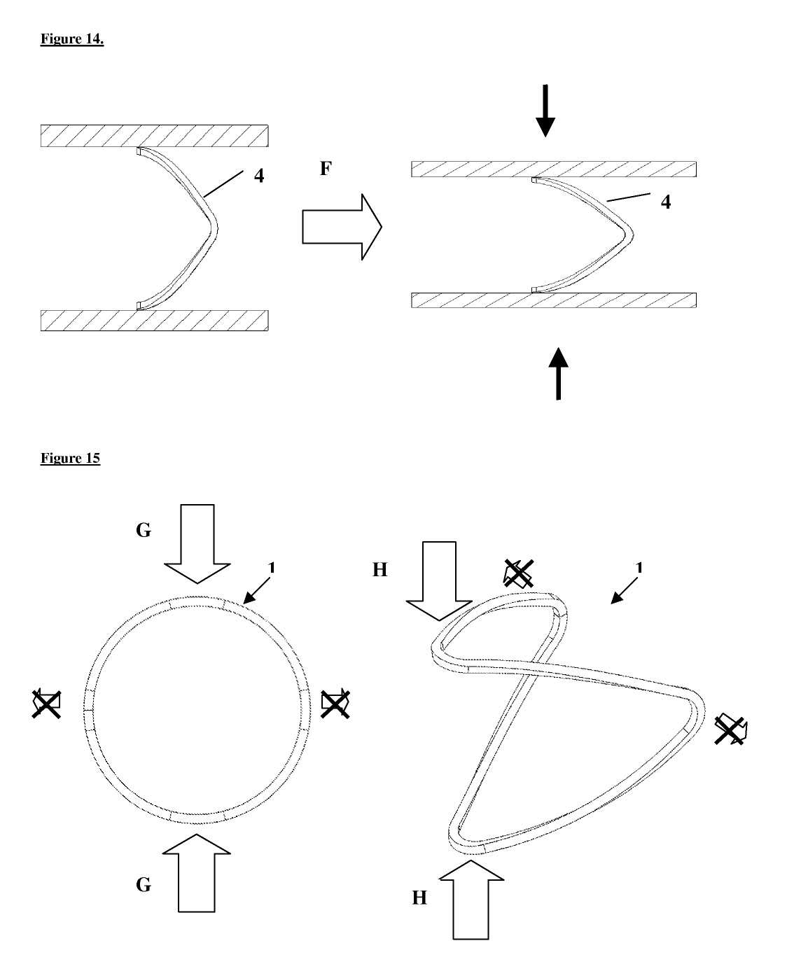

The stent of the present invention is particularly suited to use in vessels which undergo significant deformation and bending. Unlike multi-crown stent rings, when the hoop member 1 described herein is subjected to a point or crush load (indicated by arrows G and H in FIG. 15), it exerts a reduced or minimal resultant force or deformation in a perpendicular direction to the load (FIG. 15).

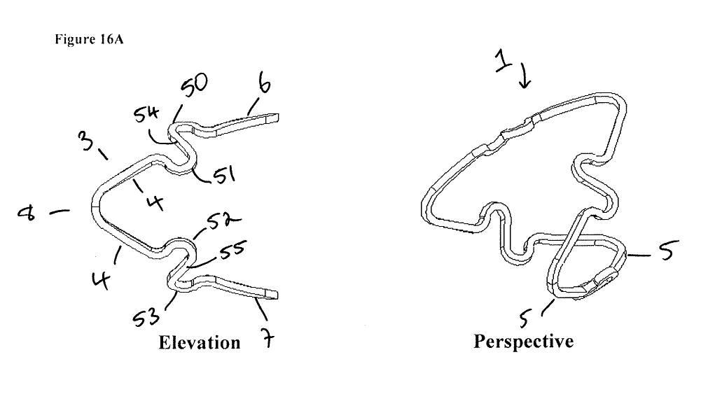

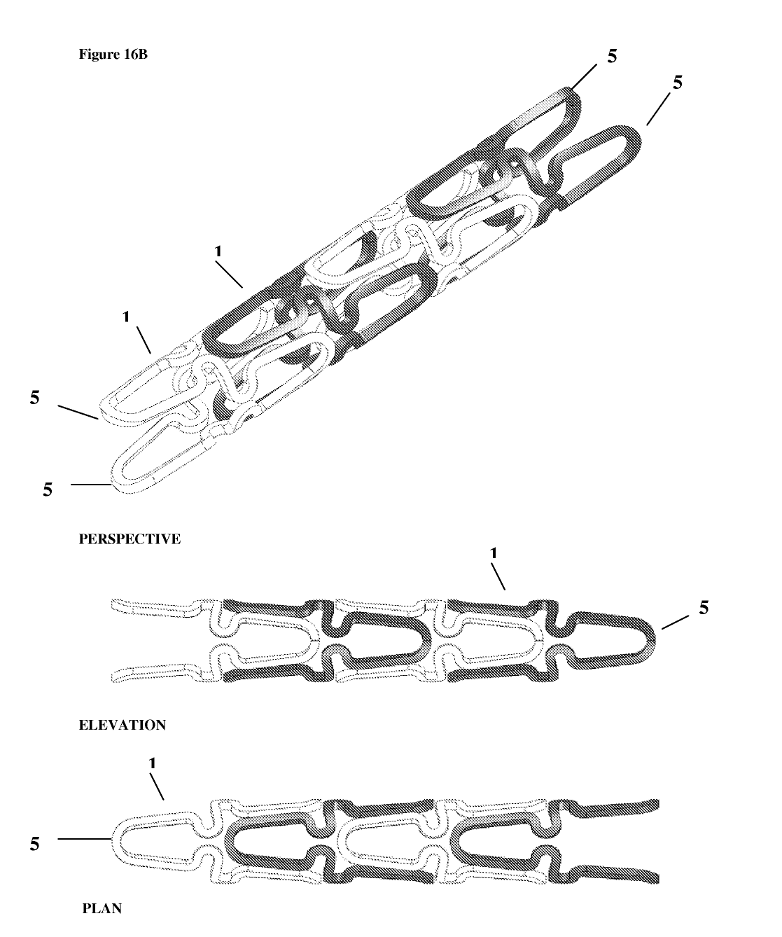

In FIGS. 16A and 16B, there is illustrated a further embodiment of the region 3 of the hoop member 1 having a variety of anfractuous or convoluted structures (see FIGS. 3 and 4). In FIG. 16A, the region 3 comprises axial struts 6,7, a curved outer end 8, and bulges 50,51,52,53 in each of axial struts 6,7. Axial struts 6,7 are joined to opposing portions 4 connecting the two regions 3 (not shown). Abutments 54,55 are located axially along struts 6,7 and between bulges 50,51 and 52,53, respectively. FIG. 16B illustrates hoop member 1 deployed as a multiple hoop member series having the geometry described for 16A. Each individual hoop members 1 are highlighted and are in the collapsed configuration prior to placement in a vessel.

The geometry of region 3 as described above and illustrated in FIG. 16A maintains the 1:1 aspect ratio while increasing blood vessel wall coverage when in an expanded configuration and can act as an abutment when in a compressed configuration loaded into a delivery system. The articulation feature along the axial struts 6,7 can improve strut bending and the ability of the hoop member 1 to conform to the vessel wall. This feature can also be used to anchor the hoop member 1 in the vessel to prevent migration. When deployed in a blood vessel as a multiple hoop member series, less hoop members 1 may be required to achieve the target length, than when region 3 is a loop structure, due to the increased blood vessel wall coverage provided by the geometry of the region 3. However the hoop member 1 still maintains the generally ‘C’ or ‘V’ shape in the plan and elevation views.

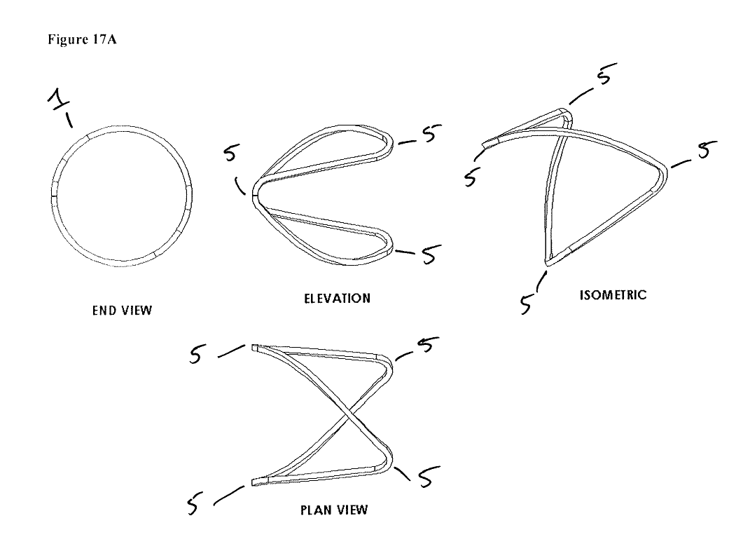

In another configuration of the hoop member 1 of the present invention, the circumferentially extending portions (or crowns) 5 are circumferentially offset on the proximal end relative to the distal end (FIG. 17A). This may improve the cylindrical deployment of the stent in the vessel and may also improve the axial elongation or compression characteristics of the stent element. In another configuration of the hoop member 1 of the present invention, the circumferentially extending portions (or crowns) 5 are axially offset on the proximal end or distal end (FIG. 17B), or in another embodiment both the proximal and distal ends are offset. This configuration could be particularly suited to improving vessel wall support in complex anatomy.

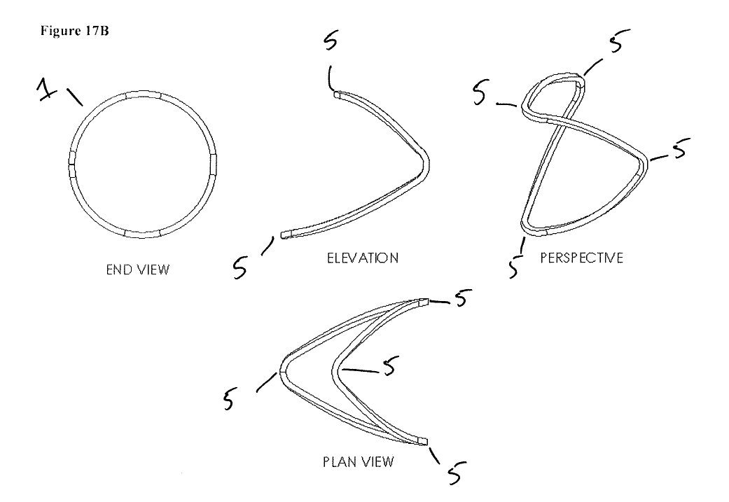

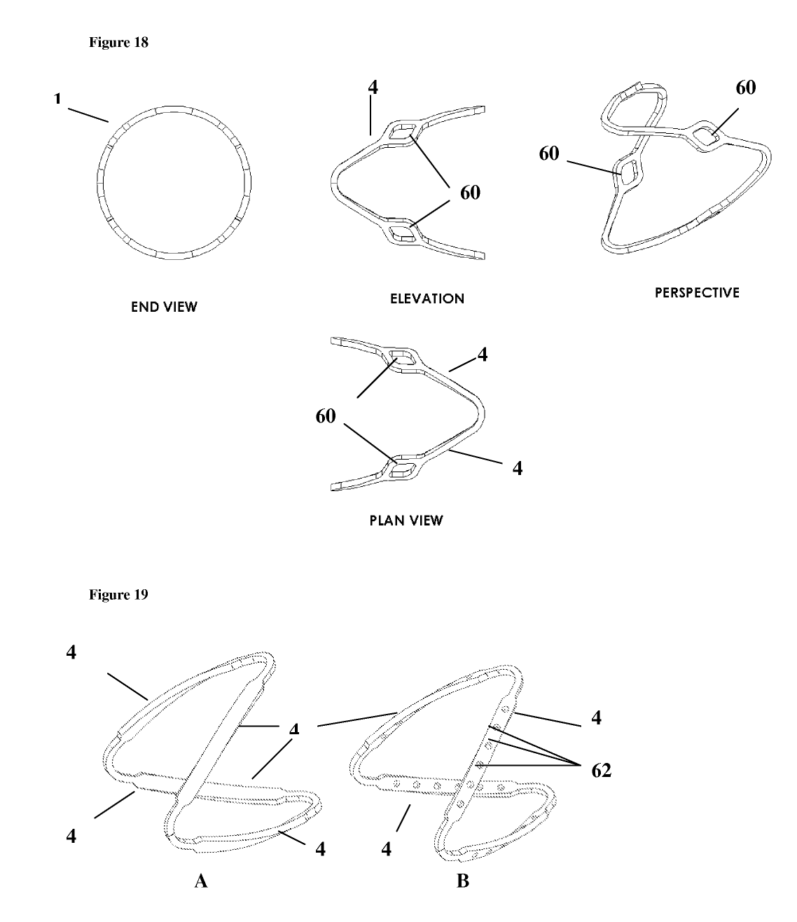

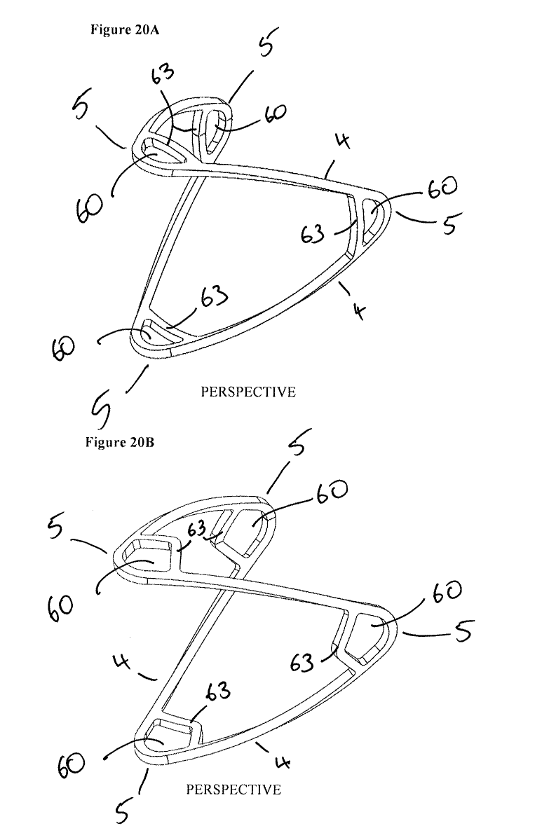

In a further embodiment of the hoop member, the hoop member 1 can be formed with a combination of closed segments 60. These closed segments 60 may be located along the opposed portions (or axial struts) 4 of the hoop member 1 (see FIG. 18) or at the generally circumferentially extending portion (or crown) 5 connecting the opposed portions 4 (see FIGS. 20A and 20B). However as before, the hoop member 1 still maintains the generally ‘C’ or ‘V’ shape in the plan and elevation views (FIG. 18). Furthermore, for each of the above-described hoop member 1 configurations, the opposed portion (or axial strut) width (for example, see FIG. 19A), wall thickness in the radial direction, surface profile (for example, a plurality of apertures 62—see FIG. 19B), and cross sectional shape can vary around the hoop member to optimise the performance characteristics. The surface texture or shape may also be optimised to facilitate different stent coatings (see FIGS. 19A and 19B).

The provision of a reinforcing element 63 linking the two axially opposed portions (or axial struts) 4 in the region of the generally circumferentially extending portions (or crowns) 5 forms closed segments 60 located at the generally circumferentially extending portions (or crowns) 5 as illustrated in FIGS. 20A and 20B. These provide additional support and strength performance characteristics to the stent of the present invention. The positioning of the closed segments 60 at the crowns 5 reinforces the structure and acts to spread the supporting surface area of the hoop member 1 without compromising its suitability for uniform and non-uniform radial force distribution.

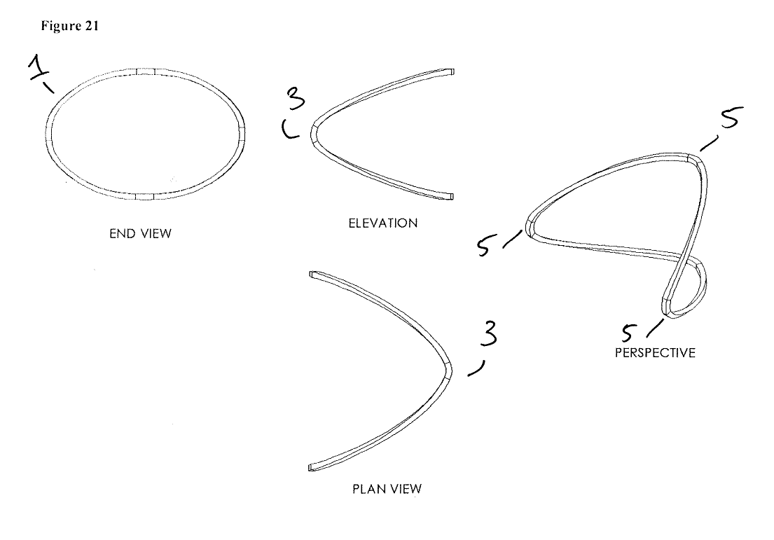

In another embodiment the hoop member 1 has an oval or non-round profile after deployment in the vessel. An example is shown in FIG. 21 where the end view (viewed along the vessel axis) of the hoop member 1 is oval. This oval shape could be beneficial in treating non-round or eccentric lesions or for specific lesion bending characteristics. The hoop member 1 of this invention is particularly suited to conforming to oval geometry because of the uniform radial force across a range of expanded diameters.

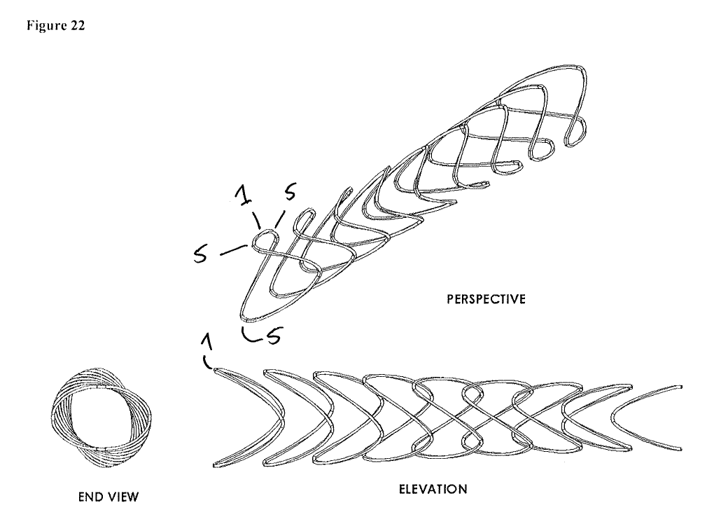

In the present invention, an entire stent containing a number of hoop members 1 as described above, can be orientated so that each hoop member is parallel and has the same orientation when deployed. Alternatively an angular displacement can be applied between each hoop member 1 when viewed axially. This angular displacement causes the hoop members 1 to form a helical configuration around the vessel centre-line (see for example, FIG. 22). The helical configuration can be a left or right helix of varying pitch depending on the spacing and rotation of the hoop members.

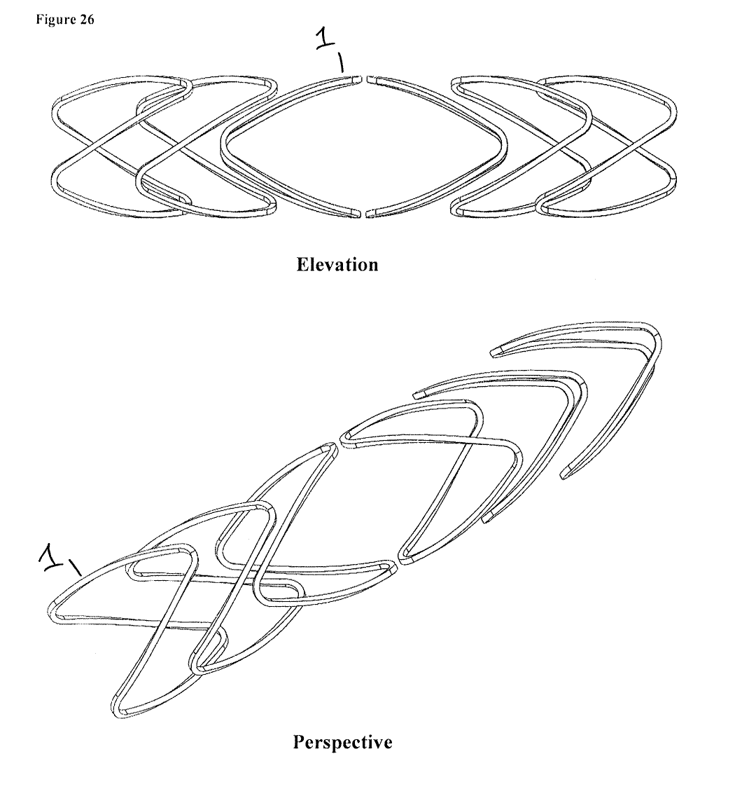

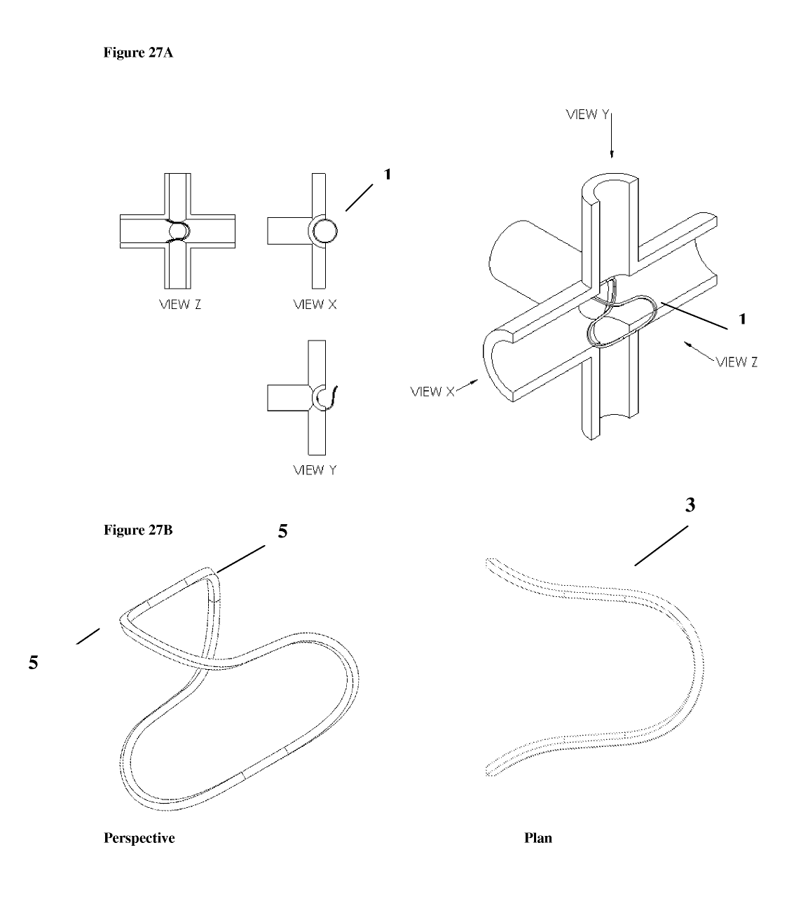

The hoop member can be configured to have a non-uniform radial force distribution. The higher radial force can then be targeted at a specific radial location/region in the vessel. Tailored radial force can also be used to remodel the cross section of the vessel to optimise blood/fluid flow velocity and pressure distribution. This may achieve a more uniform distribution of wall shear stress. Non-uniform radial force distribution may also allow for increased anchoring per opposed portions (or axial struts) 4 or per segment in the vessel (for example, see FIG. 27 described below). The non-uniform radial force distribution can be achieved within the design of a single hoop member 1 or by varying the orientation of each hoop member 1 as it is deployed in the vessel, e.g. rotating each stent ring by a fixed degree relative to the previous stent ring deployed (for example, see FIG. 22 described below).

A stent formed with multiple oval stent rings (such as those of FIG. 21) can change the cross sectional profile of the vessel lumen on deployment without significantly affecting the lumen central axis. By introducing a helical form to the vessel shape, for example by introducing a stent such as illustrated in FIG. 22, the localised fluid flow profile can be modified to reduce areas of low shear stress associated with atherosclerosis and restenosis. In another embodiment the struts of the stent rings impinge on the fluid flow in such a way as to modify the fluid flow to reduce areas of low shear stress.

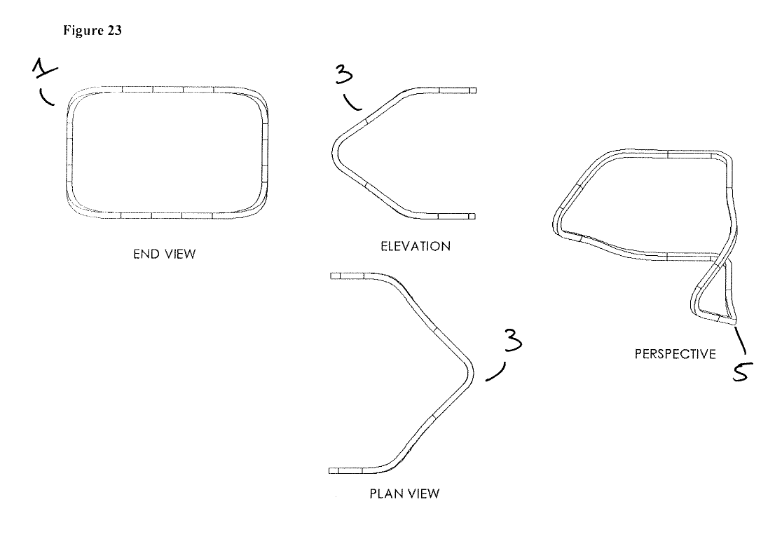

In another embodiment of the present invention, a hoop member 1 has a configuration where the profile is predominantly rectangular when viewed along the vessel axis (FIG. 23).

FIG. 24 illustrates a plurality of hoop members 1 that are collapsed for deployment into a delivery system or for insertion into a vessel. As will be understood by those skilled in the art, any of the configurations described above for each of the hoop members 1 can be collapsed in this configuration for deployment to a vessel.

The stent comprising of a number of individual hoop members 1 of the present invention can be delivered and deployed at the target location in the vessel lumen, by a number of different types of delivery systems. In one method the stent is collapsed (see FIGS. 16B or 24) and positioned in a catheter sheath which is positioned in the target location. The stent is prevented from moving proximally in the catheter and the sheath is retracted to uncover the stent and allow it to expand and contact the vessel wall. Due to the segmented nature of this stent the deployment can be stopped at any point and the catheter repositioned to deploy an additional stent at a second target location. Therefore a single catheter delivery system can be used to spot stent a number of lesions in single or multiple vessels until the full length of the constrained stent is used up.

In addition, the catheter can be moved proximally or distally during the procedure to vary the axial spacing between the deployed hoop members. This allows the radial force and vessel wall coverage to be tailored by the user during the procedure to suit the characteristics of the vessel or lesion. This movement of the distal end of the catheter to vary the axial spacing of the hoop members relative to one another can be achieved by the user manually moving the proximal end of the catheter or by a mechanism controlling the spacing, activated by the user and located in the catheter, catheter handle or device assembly. An example of a stent with variable spacing of the hoop members 1 relative to one another is illustrated in FIG. 25.

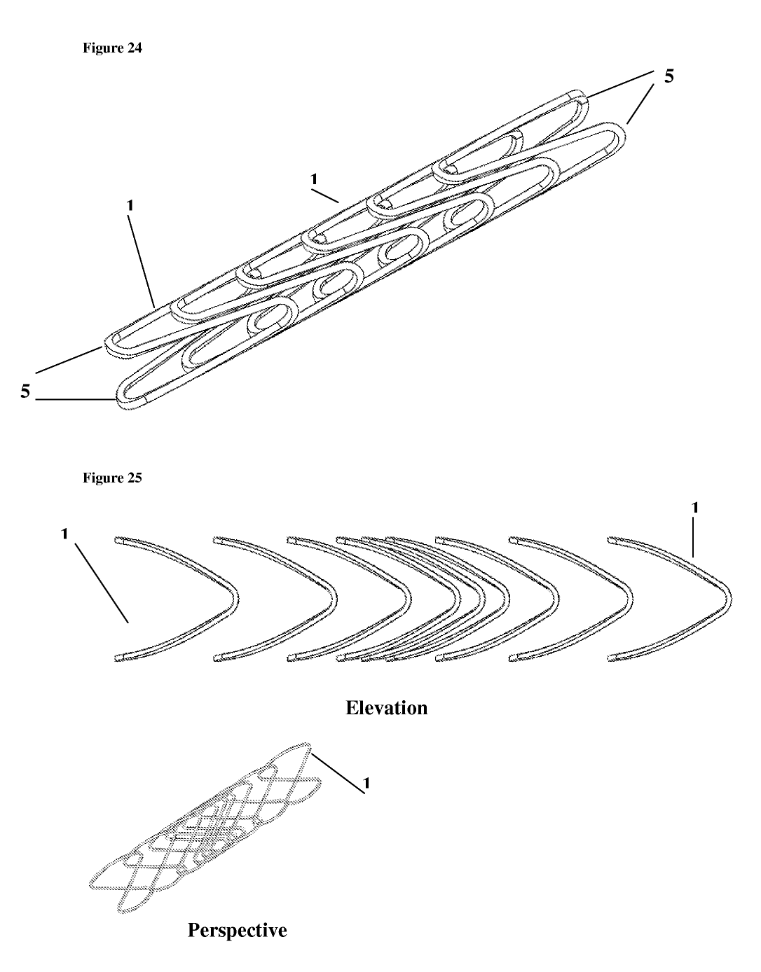

In addition to the customisation of the axial spacing, the user can also control the rotation of the hoop members 1 during the deployment. This can be achieved by the user applying a torque to the proximal end of the delivery system or by activating a mechanism contained within the catheter, catheter handle or device assembly. The ability to tailor the rotation of the hoop member 1 allows radial force and vessel wall coverage to be optimised, particularly for collateral flow. FIG. 26 is an example of where axial spacing and rotation of the hoop members 1 have been modified to give a gap for unrestricted collateral flow perpendicular to the treated vessel. Alternatively the stent comprising of a number of individual hoop members 1 can be loaded in the delivery system with preset axial and angular offsets when viewed in the axial direction. Therefore when the stent is deployed from the delivery system in the vessel lumen, the hoop members are positioned with varying axial and/or angular spacing to suit the target geometry without additional input from the clinician.

The reduced number of generally circumferentially extended portions (or crowns) 5 in the hoop member 1 of the present invention compared to those of the prior art, facilitates the rotation of each hoop member 1 relative to the previously deployed hoop member. This can allow the optimisation of radial force distribution and also the optimisation of vessel wall coverage. The vessel wall coverage can be increased at a particular target location or decreased e.g. to minimise interference of fluid flow at a collateral vessel (see FIGS. 25 and 26).

The reduced number of generally circumferentially extended portions (or crowns) 5 in the hoop member 1 facilitate a reduced crimp diameter in the collapsed configuration, therefore the stent can be crimped into a smaller diameter delivery system. Alternatively the radius of the generally circumferentially extended portions (or crowns) 5 can be increased to give a better distribution of strain energy for a fixed crimp diameter. It also allows for an increased width of the opposed portions (or axial struts) 4 and/or increased number of hoop members 1 per unit length for the same diameter of delivery system.

The stent delivery system can also be constructed so that the compressed hoop members are not only constrained by an outer sheath but are also partially or completely constrained by a connection to an inner core tube. At the target deployment site the stent is released and allowed to expand and contact the vessel wall.

Alternatively the stents can be constrained for delivery to the target location on a guide wire as described previously.

A single hoop member 1 can provide vessel support and facilitate flow in 3 axial directions (x, y, and z) with minimal interference of fluid flow (see FIG. 27A). An example of a single hoop member 1 which can be used in the 3-axis model is illustrated in FIG. 27B.

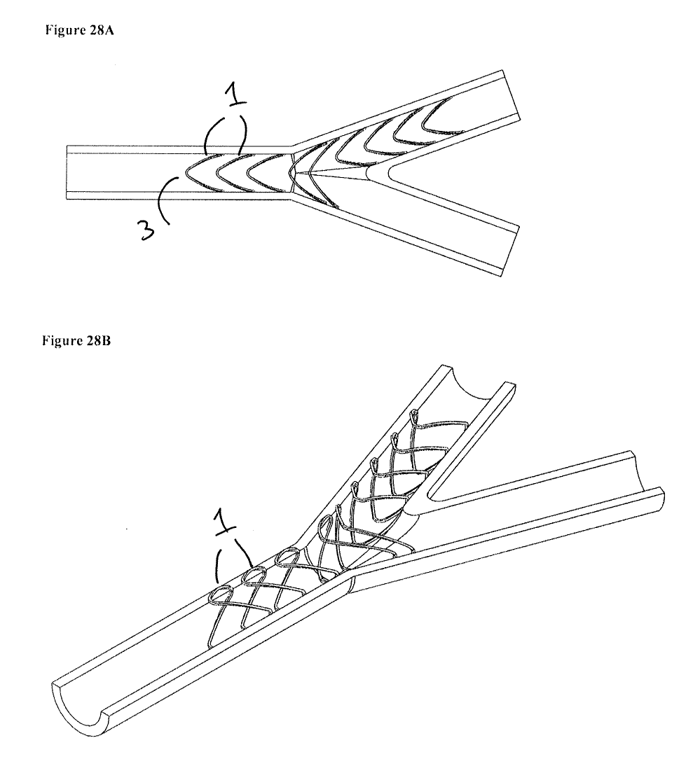

A combination of round and oval hoop members 1 as described above can be combined in a multi-ring stent to give optimal characteristics for deployment of the stent in a bifurcation or complex anatomy. A number of oval or circular hoop members 1 with different diameters can be included in the stent to give additional vessel wall coverage, radial force and stability when deployed across a bifurcation. The stent can be designed so that specific hoop members can bridge across two vessels giving improved vessel wall coverage of the lesion while maintaining flow in both vessels (see for example, FIG. 28).

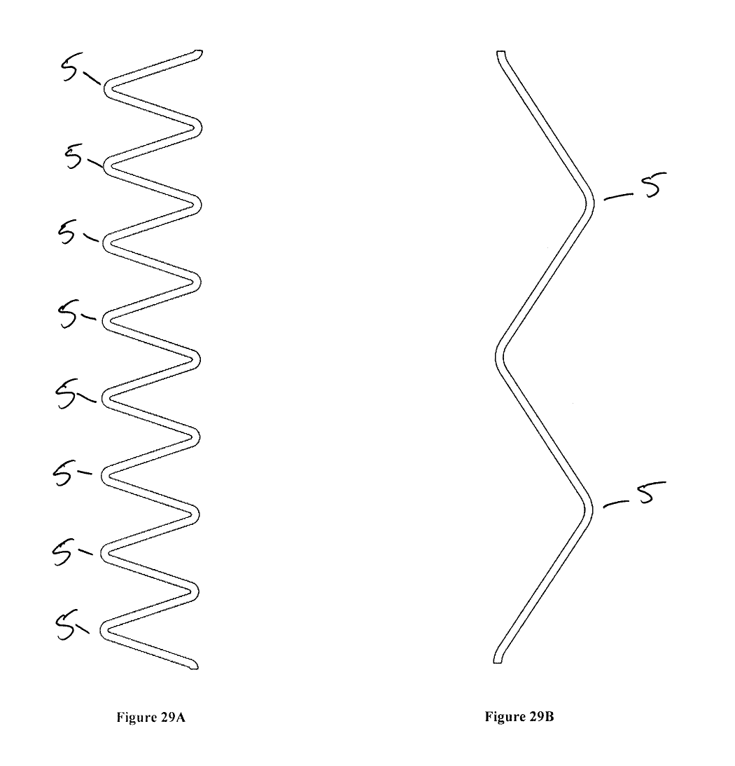

FIG. 29A shows a comparison of the articulation angle for a stent ring having eight crowns 5 at either end compared to the hoop member of the present invention having two crowns 5 at either end (FIG. 29B). Both stent rings have an aspect ratio of 1:0.5 where the axial length is half of the vessel diameter. The articulation angle is calculated with the stent ring unwrapped into a two dimensional configuration. The prior art stent design has a typical articulation angle of 20 to 60°. The individual hoop member of this patent has an articulation angle greater than 60°.

Similarly the stent can include combinations of various shapes and sizes of hoop members 1 for deployment in specific complex geometries such as, but not limited to, trifurcations, aneurysms, plus complex lesion and vessel shapes. The hoop members 1 can conform to complex sizes and shapes and single or multiple lumens. As the hoop members are unconnected, any of the hoop members can expand into complex shaped anatomy providing improved vessel support.

The embodiments of this invention described above that consist of a hoop member 1 without any closed cells are particularly safe for implanting in patients who undergo subsequent Magnetic Resonance Imaging (MRI). The impact of MRI on the segmented and open cell nature of this stent is reduced compared to standard designs of non-segmented stents.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. A stent comprising:
at least one individual closed hoop member formed from a resilient material, the hoop, in a two dimensional configuration, having a diametral dimension that exceeds the diameter of the largest blood vessel into which it will be inserted; and
the hoop being resiliently deformable or resiliently deformed so as to occupy a spatial envelope of reduced diameter when inserted in a lumen,
wherein when occupying the spatial envelope of reduced diameter, the closed hoop member has at least two substantially diametrically-opposed generally circumferentially extending regions; and
at least two regions that extend at least in part generally axially relative to the generally circumferentially extending regions,
each of said regions that extend at least in part generally axially being disposed between two of said generally circumferentially extending regions.

2. A stent according to claim 1, wherein:
each of said regions that extend at least in part generally axially has:
at least two opposed portions that extend generally axially with respect to said plane substantially defined by the generally circumferentially extending regions, and
a generally circumferentially extending portion connecting said opposed portions.

3. A stent according to any of claim 1 or claim 2, wherein:
each of said at least two regions that extend at least in part generally axially with respect to the plane substantially defined by the generally circumferentially extending regions extend in the same axial direction.

4. A stent according to any of claims 1 to 3, wherein:
one of said at least two regions that extend at least in part generally axially with respect to a plane substantially defined by the generally circumferentially extending regions extends in the opposite axial direction from the other of said at least two regions that extend at least in part generally axially.

5. A stent according to any of claims 1 to 4, wherein:
the hoop member is configured for expansion from a compressed fitting disposition in which said at least two regions that extend at least in part generally axially are at a first transverse spacing from one another to a second expanded disposition of use in which said at least two regions that extend at least in part generally axially are at a second transverse spacing from one another, the second spacing being greater than the first spacing.

6. A stent according to claim 5 together with compression means for constraining at least one hoop member in said compressed fitting disposition for placement of the stent within a blood vessel, the compression means being subsequently removable to allow the hoop members to expand individually, each to a respective second expanded disposition in which axial and circumferential regions of the hoop member engage against the inner wall of the blood vessel.

7. A stent as claimed in any preceding claim wherein each hoop member has two crowns at the proximal end and two crowns at the distal end of the stent.

8. A stent according to any preceding claim wherein an articulation angle is selected such that the increase in radial force per unit length delivered onto the lumen equals or exceeds the decrease in radial force per unit length associated with the strain energy within the stent material so that the hoop member shortens axially with radial expansion.

9. A stent according to claim 8 where the articulation angle is in the range 60° to 170°.

10. A stent as claimed in any preceding claim wherein at least one pair of the generally axially opposed regions are arranged with an axial separation between them.

11. A stent as claimed in any preceding claim which is formed with a non-circular end view profile in its non-compressed state.

12. A stent according to any preceding claim wherein the generally circumferentially extending regions are U-, C- or V-shaped.

13. A stent as claimed in any preceding claim which has an oval or non-round profile after deployment in the vessel lumen.

14. A stent according to any of claim 2 to claim 13 wherein the generally circumferentially and/or axially extending portion has a geometry selected from the group comprising loop, anfractuous, and crenellated structures.

15. A stent according to any preceding claim wherein the hoop member is provided with an anchoring means which is adapted to anchor the stent against the lumen wall.

16. A stent according to any of the preceding claims wherein the hoop profile has an aspect ratio of 1:1 when occupying a spatial envelope of reduced diameter for insertion into a blood vessel or when expanded in a blood vessel.

17. A stent according to any one of the preceding claims wherein the series of axially arranged hoop members consist of from 1 to 400 hoop members.

18. A stent according to claim 17 wherein the axial distance between individual hoop members in a series of axially arranged hoop members is in the range of 0 mm to 20 mm in situ.

19. A stent according to claim 14 wherein the overall length of the axially arranged series of hoop members is in the range of from 1 mm to 400 mm.

20. A stent according to any one of the preceding claims wherein at least one hoop member is nested on a wire, mandrel or guide wire.

21. A stent according to any preceding claim wherein at least one hoop member is nested on a wire, mandrel or guide wire and encapsulated by a sheath constraining the hoop members.

22. A stent according to any preceding claim wherein the hoop member is plastically deformable and/or balloon expandable.

23. A stent according to any of the preceding claims wherein a series of hoop members are axially arrangable offset to each other on opposite sides of a central circumferentially defined plane.

24. A stent according to any of the preceding claims wherein a series of hoop members are deployable so that a pair of hoop members crosses each other providing a maximum of 4 points of contact with each other.

25. A stent according to any of the preceding claims wherein the resilient material is selected from the group comprising metals, plastics, ceramics or composites thereof.

26. A stent according to claim 25 wherein the resilient material is selected from the group comprising shape memory alloys such as copper-zinc-aluminum-nickel, copper-aluminum-nickel, iron-manganese-silicon, and nickel-titanium (NiTi) alloys or shape memory polymers manufactured using multi block co-polymers synthesised from MDI/1,4-butanediol, poly(ε-caprolactone), poly(tetrahydrofuran), poly(ethylene adipate), poly(ethylene terephthalate), poly(ethylene oxide), poly(1,4-butadiene), Polyethylene, poly(vinyl acetate), polyamide-6 (nylon-6), or, poly(2-methyl-2-oxazoline).

27. A stent as claimed in any of claims 1 to 26 wherein the hoop member is formed from a biodegradable material selected from the group comprising Poly-L-lactic acid (PLLA), polyglycolic acid (PGA), poly(D,L-lactide/glycolide) copolymer (PDLA), polycaprolactone (PCL), magnesium, or any other biodegradable material.

28. A stent according to any of the preceding claims wherein the hoop member is coated with a biodegradable material selected from the group comprising Poly-L-lactic acid (PLLA), polyglycolic acid (PGA), poly(D,L-lactide/glycolide) copolymer (PDLA), polycaprolactone (PCL), magnesium, or any other biodegradable material.

29. A stent according to any preceding claim wherein the wherein the hoop member acts as a carrier for a drug to be delivered to the blood vessel.

30. A stent according to any of claim 1 to claim 29 wherein the hoop member is coated with a bioabsorbable drug eluting material.

31. A stent as claimed in any preceding claim in which the geometry of the hoop members 1 when positioned in a lumen induces a uniform distribution of blood vessel wall shear stress, without significantly distorting the lumen central axis.

32. A stent as claimed in any preceding claim provided with a reinforcing element linking the two axially opposed portions (or axial struts) in the region of the generally circumferentially extending portions (or crowns).

33. A kit of parts comprising at least one hoop member as claimed in any of claims 1 to 32 mounted within a sheath, or mounted on a wire, mandrel or guide wire.

34. A kit of parts comprising a plurality of hoop members mounted along a sheath or wire or mandrel or guide wire at varying axial spacings, or axially off-set with respect to one another or at varying angular spacings when viewed in the axial direction such that they can be deployed at varying spacings or in varying orientations within a blood vessel.

35. A stent substantially as described herein with reference to the accompanying drawings.
